SEQUENCE LISTING (11 sequences)

SEQ ID NO: 1 - protein, 496 aa, homo sapien
QYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 2 - protein, 496 aa, Homo sapiens
QYSPNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMSGNAVSAGTSSTCGSYFNPGSRDFPAVPYSGWDFNDGKCKTGSGDIENYNDATQVRDCRLVGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 3 - protein, 511 aa, homo sapien
MKLFWLLFTIGFCWAQYSSNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLSGLLDLALGKDYVRSKIAEYMNHLIDIGVAGFRIDASKHMWPGDIKAILDKLHNLNSNWFPEGSKPFIYQEVIDLGGEPIKSSDYFGNGRVTEFKYGAKLGTVIRKWNGEKMSYLKNWGEGWGRYFENGKDVNDWVGPPNDNGVTKEVTINPDTTCGNDWVCEHRWRQIRNMVNFRNVVDGQPFMPSDRALVFVDNHDNQRGHGAGGASILTFWDARLYKMAVGFMLAHPYGFTRVMSSYRWPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 4 - protein, 511 aa, homo sapien
MKLFWLLFTIGFCWAQYSSNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLSGLLDLALGKDYVRSKIAEYMNHLIDIGVAGFRIDASKHMWPGDIKAILDKLHNLNSNWFPEGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRYFENGKDVNDWVGPPNDNGVTKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 5 - protein, 511 aa, homo sapien; identical to SEQ ID NO: 4

SEQ ID NO: 6 - protein, 254 aa, homo sapien
MKFFLLLFTIGFCWAFKYGAKLGTVIRKWNEKMSYLKNWGEGWGFVPSDRALVFVDNHDNQRGHAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 7 - protein, 496 aa, homo sapien
QYSSNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLSGLLDLALGKDYVRSKIAEYMNHLIDIGVAGFRIDASKHMWPGDIKAILDKLHNLNSNWFPEGSKPFIYQEVIDLGGEPIKSSDYFGNGRVTEFKYGAKLGTVIRKWNGEKMSYLKNWGEGWGRYFENGKDVNDWVGPPNDNGVTKEVTINPDTTCGNDWVCEHRWRQIRNMVNFRNVVDGQPFMPSDRALVFVDNHDNQRGHGAGGASILTFWDARLYKMAVGFMLAHPYGFTRVMSSYRWPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 8 - protein, 496 aa, homo sapien
QYSSNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLSGLLDLALGKDYVRSKIAEYMNHLIDIGVAGFRIDASKHMWPGDIKAILDKLHNLNSNWFPEGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRYFENGKDVNDWVGPPNDNGVTKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 9 - protein, 496 aa, homo sapien; identical to SEQ ID NO: 8

SEQ ID NO: 10 - protein, 496 aa, homo sapien; identical to SEQ ID NO: 1

SEQ ID NO: 11 - protein, 496 aa, homo sapien; identical to SEQ ID NO: 2

(19) United States
(12) Patent Application Publication
(10) Pub. No.: US 2013/0273026 A1
(43) Pub. Date: Oct. 17, 2013

(54) COMPOSITION AND METHOD FOR MODULATING INFLAMMATORY MOLECULES WITH AMYLASE

(71) Applicant: INTEGRATIVE ENZYMATICS, INC., Santa Fe, NM (US)

(72) Inventors: Madhavi Gavini, Cambridge, MA (US); Raja Srinivas, Cambridge, MA (US)

(21) Appl. No.: 13/862,252

(22) Filed: Apr. 12, 2013

Related U.S. Application Data
(60) Provisional application No. 61/623,485, filed on Apr. 12, 2012.

(51) Int. Cl. A61K 38/47

(52) U.S. Cl. CPC A61K 38/47
USPC 424/94.61; 435/375; 435/201

(57) ABSTRACT

A method and composition for treating in a mammalian subject a condition accompanied or caused by IgE mediated histamine release from mast cells comprising administering to a subject in need of such treatment a therapeutically effective amount of the pharmaceutical composition an amylase peptide or derivative thereof.

### CROSS-REFERENCE TO RELATED APPLICATIONS

This application claims priority to and the benefit of the filing of U.S. Provisional Patent Application No. 61/623485, entitled “Composition and Method for Modulating Inflammatory Molecules with Amylase”, filed on Apr. 12, 2012, and the specification and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable

### BACKGROUND

Type I Diabetes Mellitus (T1DM) poses a serious challenge to healthcare in the United States. It is a disease characterized by the autoimmune destruction of the insulin-producing pancreatic β-cells, resulting in an inability to regulate blood glucose levels. The only currently approved treatment for individuals with T1DM is life-long, regular insulin injections. While such a regimen can be capable of effectively controlling blood glucose levels, it does not guarantee the prevention of largely irreversible secondary complications. Among the most common of these secondary complications are cardiovascular disease, diabetic retinopathy, and diabetic nephropathy.

Cardiovascular disease (CVD) represents the leading cause of morbidity and mortality in afflicted patients. However, all of the secondary complications are serious impairments to the normal health and quality of life of diabetics. Currently, 1.29 million people in the United States and 17 million people worldwide suffer from this disease. Compounding the problem, the incidence rate is estimated to be growing 3-4% annually with a rapidly decreasing age of onset, such that an alternative name for the disease is considered Juvenile Onset Diabetes (Atkinson et al. 2001).

Traditionally, diabetes has been classified mainly as a disorder of metabolic homeostasis. However, recent research has strongly implicated chronic inflammation as one of the major pathological side effects. Multiple epidemiological studies and clinical trials have indicated that Type I diabetics often suffer from chronic, low-grade inflammation (Mangge et al. 2004 and Pietropaolo et al. 2007). This inflammation is speculated to be the leading cause behind the eventual autoimmune destruction of the beta-cells in T1DM patients. Recent research has revealed that sustained inflammation is also central to the etiology of the associated vascular complications.

The inflammatory cascade contributing to the development of CVD has been rapidly elucidated over the past decade, inspired by the marked increase in disease prevalence. To put this in perspective, nearly 70% of all T1DM fatalities are attributed to the condition. In essence, altered cytokine and cellular adhesion molecule expression is thought to enhance the recruitment and alter the activities of leukocytes involved in the inflammatory pathogenesis of the disorder.

In general, cytokines are powerful signaling molecules, involved in both local and systemic modulation of inflammation. While the exact physiological effects of pro-inflammatory cytokines have not been clearly delineated, they have demonstrated a clear predictive value for vascular complications and CVD. In particular, studies have established that patients with elevated basal levels of IL-6 and TNF-alpha suffer an increased risk for a future cardiovascular event (Libby et al. 2002).

Diabetic retinopathy (DR), in contrast to CVD, is a microvascular complication that affects the vessels of the eye supplying the retina. A study examining proliferative DR found that both vitreous humor and serum levels of IL-1β and TNF-alpha were elevated when compared to the controls (Demircan et al. 2006). Additionally, an experimental study conducted by Joussen et al. (2002) found that the therapeutic effect of nonsteroidal anti-inflammatory drugs (NSAIDs) on DR was in part mediated by the suppression of the cytokine, TNF-alpha. However, NSAIDs are not typically used as a treatment because at the therapeutic dosage necessary, they cause many harmful side effects. Nevertheless, this study demonstrates that suppression of TNF-alpha can have ameliorating effects in the treatment of vascular complications associated with DR.

Similar results have been observed in studied cases of diabetic nephropathy, which have been marked by increased basal levels of certain cytokines (TNF-alpha, IL-6, IL-1) and experimental treatments focused on modulating these same markers. Multiple studies have revealed that levels of cytokines in serum and urine are positively correlated with the progression of the disease. Particularly related to the pathogenesis of nephropathy, molecules such as IL-1 and IL-6 have been identified as being responsible for the altering of the permeability of vascular endothelial cells and the development of basement membrane thickening respectively (Dronavalli et al. 2008).

Another group of molecules commonly found in elevated levels in diabetics are advanced glycation end (AGE) products. These are species resulting from the non-enzymatic glycosylation and various other chemical modifications to proteins and lipids. While many of the intermediates in the formation of AGEs are toxic to cells, most of the ongoing research has explored the AGE molecules interactions with the receptor for AGE (RAGE) immunoglobulin superfamily of peptides. AGE-RAGE interaction is speculated to be heavily involved in pro-inflammatory processes. Two conditions commonly found in diabetic patients, hyperglycemia and oxidative stress, have been shown to increase the rate of formation of AGEs. Thus, it is speculated that these molecules represent another source of chronic inflammation in diabetic individuals.

Beyond their RAGE mediated effects, AGE also seem to affect the activity of the IgE antibody. A study in 2001 observed increased binding of advanced glycation end products to the immunoglobulin (Chung and Champagne, 2001). This enhanced affinity suggests a potential for a higher allerginicity of the antibody in individuals with elevated levels of AGE.

Type I diabetes is caused by the autoimmune destruction of the beta-cells of the pancreas, resulting in a disruption of normal glucose homeostasis. Elevated levels of cytokines and interleukins are implicated in beta-cell destruction and play a role in the development of common diabetic secondary complications such as CVD, DR, and nephropathy.

Amylase is an enzyme that catalyzes the hydrolysis of alpha (1-4) glycosidic linkages found in starch. It exists as two, highly homologous isoenzymes present in either salivary or pancreatic secretions. Both forms consist of a single polypeptide chain consisting of 496 amino acids, encoding for proteins with molecular weights around 55 to 60 kDa. Amylase requires both Ca²⁺ and which act as a cofactor and allosteric activator, respectively.

In the body, the pancreatic form of the enzyme is released into the intestinal lumen where it hydrolyzes the pre-digested starch oligomers into smaller oligosaccharides. This form of Amylase is a digestive enzyme that is responsible for the cleavage of certain glycosidic bonds and sugars, allowing individuals to digest sugar and various carbohydrates.

Patients with pancreatic related diseases as well as metabolic disorders commonly exhibit a chronic underproduction of digestive pancreatic enzymes, Amylase included. Current treatment regimens for individuals suffering from these diseases are often prescribed supplementation therapies that involve administration of porcine derived pancreatic enzymes. However, the use of porcine derived pancreatic enzymes is an insufficient treatment. According to bioinformatic analyses, the structures of porcine and human Amylase are different at several key allosteric and sugar binding sites. While the porcine-derived enzymes are capable of digesting food, the different structure suggests that they are not a viable substitute for human enzymes with respect to metabolic pathway regulation.

Amylase is involved in insulin regulation. The connection between insulin and Amylase has been well-studied: insulin regulates the expression of Amylase gene (Boulet et al. 1986, Johnson et al. 1993, Soling et al. 1972), Insulin deficiency is strongly correlated with decreased pancreatic Amylase levels in many types of animal models of hyperinsulinemia and insulin resistance (Trimble et al. 1986). While the literature is largely inconclusive on this point in humans, the trend seems to hold in individuals who suffer from metabolic and pancreatic exocrine deficiency related diseases (Aughsteen et al. 2005, Dandona et al. 1984, Frier et al. 1978, Nakajima et al. 2011a, Nakajima et al. 2011b, Swislocki et al 2005)

Secretion of insulin occurs in beta-cells of the pancreas in a biphasic manner (Bratanova-Tochkova et al. 2002 and Wilcox 2005). The first phase is a glucose-mediated secretion that rapidly causes the release of insulin containing granules in the cell. This first phase terminates approximately 10 minutes after release, after which a second phase is responsible for insulin secretion. While the exact mechanism is unknown, an increase in intracellular calcium and several glycoproteins (VIP, PACAP, GLP-1, and GIP) appear to play significant roles in the signaling cascade and are characteristic of this second phase (Bratanova-Tochkova et al. 2002). We postulate that Amylase may play a role in modulating this second phase by acting in a negative feedback loop with insulin and by interacting with these glycoproteins.

IgE is known to mediate the release of histamine (Becker et al. 1973, Ishizaka et al. 1970, Segal et al. 1977, and Yoo et al. 2010). A decrease in histamine is able to inhibit insulin through the inhibition of P-selectin and P Selectin Glycoprotein Ligand-1 (PSGL-1) (Snapp et al. 1998).

P-selectin is a cell adhesion molecule that is found in the interior of endothelial cells and on activated platelets. When endothelial cells are exposed to histamine, P-selectin migrates to the exterior of the cell where it inserts into the plasma membrane (Cleator et al. 2006 and Thurmond 2010). There, P-selectin mediates adhesive events that occur during inflammation, particularly the interaction between blood cells and the endothelium cells at the site of inflammation (Snapp et al. 1998). It does this through binding with PSGL-1, P-selectin's ligand located on leukocytes. Upon binding, the white blood cell is able to infiltrate the endothelial cell at the site of inflammation where it contributes to chronic inflammation (Kalupahana et al. 2012, Russo et al. 2010m and Santilli et al. 2011). Direct inhibition of PSGL-1 and P-selectin results in decreased insulin resistance (Russo et al. 2010 and Sato et al. 2011).

Deficient Amylase production is the result of a combination of environmental, dietary and genetic factors. One possible dietary factor that has been implicated is excessive glucose. Benkel et al. (1986). found that excessive glucose in drosophila inhibits Amylase quantity, not the enzymatic activity. Similarly, Nakajima et al. (1970) found that in dogs, glucagon and D-glucose inhibited exocrine pancreatic secretion and Danielsson (1973) observed similar results for glucose in mouse pancreatic beta-cells. We propose that these factors inhibit expression of the Amylase protein, either through direct damage to the acinar cells, the alpha-Amylase gene, or down regulation of the gene. This theory would resolve the paradoxical results from studies concerning the lack of serum Amylase in the presence of hyperinsulinemia and offers an explanation as to why a decrease in serum Amylase levels leads to insulin resistance.

Modulatory pathways are highly specific and even slight alterations to protein structure can entirely prevent or reduce the kinetics of a metabolic process so greatly that it is no longer functionally efficient. We believe that this explains why current Amylase supplementation therapies are insufficient for deficiencies and why supplementation might aid individuals with hyperinsulinemia. The porcine Amylase enzymes that are currently used are structurally different than human Amylase. This difference between species is even mentioned as a potential explanation to the disparate and often conflicting data that is found in research into the pancreas (Barreto et al. 2010).

Cystic Fibrosis (CF) is an autosomal genetic disorder caused by a mutation in the CF transmembrane conductance regulator (CFTR) gene that affects sodium transport and particularly affects the lungs. It is the most common life shortening genetic disorder in Caucasians of European descent, affecting 1 in 3,000 people with a median survival age of 38. Incidence of CF is much less frequent in other ethnic groups. There are approximately 30,000 individuals with CF in the United States and 70,000 worldwide. The mutation in CFTR results in a defective cAMP-regulated chloride channel that affects trans epithelial ion flow in the airways leading to problems with mucociliary clearance. As mucociliary clearance is the primary defense mechanism of the airways against infection, reduced clearance compromises host defense. This leads to colonization of the host's lung by various opportunistic bacteria where the altered mucous promotes growth and chronic infection. For reasons not entirely certain, individuals with CF are only colonized by a few select species of bacteria, the most frequent of which being Pseudomonas aeruginosa.

The sustained bacterial infection of the airways ultimately leads to the development of chronic inflammation, which is the major cause of the pulmonary disease and respiratory failure and is responsible for 80% of CF deaths.

The inflammation is characterized by an intense neutrophilic response that is mediated primarily by IL-8 released by endothelial cells. IL-8 acts as the principal chemoattractant for neutrophils in lungs of CF patients. Once the neutrophils arrive at the site of inflammation, other factors promote their migration into sub-epithelial tissue where they further release pro-inflammatory cytokines and neutrophil chemoattractants, amplifying the cycle through a positive feedback mechanism. Increases in other pro-inflammatory factors such as TNF-alpha, IL-1, IL-6 and NF-kB have been implicated in the pathogenesis as well as decreases in anti-inflammatory factors such as IL-10 and lipoxin.

While results are inconclusive, there is sizeable body of evidence that chronic inflammation might occur directly as a byproduct of the CFTR gene mutation and preceding bacterial infection. This has been demonstrated by observation of inflammation in patients that were culture negative to bacterial infection, though it may simply be explained by the heterogeneity of inflammation observed within the lung. Other studies have shown the CF cell-lines have elevated levels of NF-kB (a regulator involved with the immune response and inflammation) activation in airway epithelial cells when compared to identical non-CF cell-lines and that this activation is dependent on CFTR trafficking and chloride ion channel function.

This “pre-existing” inflammation independent of infection might, in part, explain why individuals with CF not only suffer from chronic inflammation in the airways, but also display a disproportionately active inflammatory response to infection. As measured by neutrophil concentration in the airways, the inflammatory response is ten times greater in CF patients when compared to controls. This supports the idea that individuals with CF have excessive inflammatory response and impaired inflammatory control.

Another important component of the chronic inflammatory response is the role of Mast cells. Mast cells are commonly found in and near epithelial tissues where they assist the immune system in mounting an inflammatory response. This is mediated through the release of stored factors such as histamine, TNF-alpha, IL-6, IL-1β, IL-1 and other pro-inflammatory cytokines from granules that when released help coordinate the inflammatory response by attracting leukocytes, neutrophils and by inducing inflammation directly in epithelial cells. The histamine and TNF-alpha released by the Mast cells leads to rapid expression of P- and E-selectin on the epithelial cells, which are critical in recruiting the circulating neutrophils. While Mast cells have many triggers that cause degranulation, the most prominent is the binding of immunoglobulin E (IgE). Additionally, IgE is known to bind to its receptor FccRI, which is found on mast cell as well as on macrophages, neutrophils, basophils and monocytes and induces degranulation and release of pro-inflammatory factors. 51% of patients with CF have been shown to have elevated serum levels of IgE.

While dysfunction of the lungs and airways is the hallmark of CF, other organ systems are damaged in individuals with the disease. In particular, the pancreas is severely affected with 85% of CF patients having pancreatic damage at birth and a progressive loss of function over time. The most common clinical manifestation of this is exocrine pancreatic enzyme insufficiency, which is the decrease in digestive enzymes (lipase, Amylase, and trypsinogen) and results in malnutrition. The exocrine enzyme insufficiency is impaired due to lowered digestive enzyme release and bicarbonate secretion. This has an amplifying effect where decreased digestive enzyme levels lead to inadequate levels of bicarbonate which then, leads to a sub-optimal pH for enzyme function. To overcome this deficiency, pancreatic digestive enzyme supplements have to be administered.

Damage to the pancreas is thought to be caused by accumulation of secreted materials within pancreatic ducts that lead to degradation and destruction of the acinar, part of the exocrine pancreas where digestive enzymes are produced. The CFTR gene expressed here, and the mutation of the gene is related to the severity of pancreatic disease.

Alpha-Amylase is one of the digestive enzymes secreted by the body. It catalyzes the hydrolysis of alpha-1,4 glucan linkages in starches and other polysaccharides. Human alpha-Amylase contains 496 amino acids in a single chain and is encoded by one of two genes: AMY1 and AMY2 (Ferey-Roux at al. 1998). These two genes correspond to the two major isoforms of the enzyme; AMY1 encodes for salivary alpha-Amylase, which is secreted in the mouth by the salivary glands, and AMY2, which encodes for pancreatic alpha-Amylase, is secreted by the pancreas and found in the duodenum (the first section of the small intestine). Both forms of alpha-Amylase are calcium-requiring metallo enzymes, requiring a single calcium ion and chloride ion for full enzymatic activity (Whitcomb and Lowe 2007). However, while both share significant similarity, there are distinct differences between the two: amino acids near the active site, glycosylation sites, the isoelectric point, and the optimum pH are all different (Zakowski and Bruns 1985). Despite these differences, though, both isoforms of alpha-Amylase are found circulating within the blood serum (Berk et al. 1966 and Fridhandler et al. 1972).

Current treatments of CF are based around four treatment options: addressing the malnutrition, relieving obstruction of the airway, treatment of the infections in the airway, and suppression of inflammation. To address the malnutrition, supplements of pancreatic exocrine digestive enzymes are used. Drugs are prescribed to enhance mucous clearance. Antibiotics are used for neutralizing the chronic bacterial infection

No current, commonly prescribed treatment for inflammation is effective. High dosages of ibuprofen and steroids have been used to suppress inflammation but the side effects (ibuprofen can cause gastrointestinal hemorrhage and the steroids cause stunted growth, cataracts, and diabetes) are considered undesirable enough that regular usage is not advised. Many scientists agree that better alternatives for suppression of inflammation are needed to augment current therapy.

Note while a discussion refers to a number of publications by author(s) and year of publication, and that due to recent publication dates certain publications are not to be considered as prior art vis-a-vis the present invention. Discussion of such publications herein is given for more complete background and is not to be construed as an admission that such publications are prior art for patentability determination purposes.

### BRIEF SUMMARY

One embodiment of the present invention provides for a method of modulating IgE mediated histamine release from an IgE receptor positive cell capable of releasing histamine in-vitro or in-vivo wherein an effective dose of an Amylase peptide or a derivative thereof is provided to the IgE receptor positive cell in-vitro or in-vivo under conditions that would permit binding of Amylase to free IgE in solution to form an IgE-Amylase binding pair thereby inhibiting the binding of free IgE to the IgE receptor positive cell. In a preferred embodiment the cell is a mast cell, a basophil or an antigen-presenting dendritic cell. In a preferred embodiment the Amylase peptide is pancreatic alpha-Amylase. For example the Amylase peptide is selected from SEQ ID NO 1-11 or a derivative thereof. In another preferred embodiment the Amylase peptide derivative is a composition having at least 90% sequence homology with amino acids 417-427 of SEQ ID NO. 1 and at least 70% sequence homology with the remaining amino acids of SEQ ID NO 1.

Another embodiment provides a method of treating Type I diabetes or Type II diabetes in a mammalian subject wherein a therapeutically effective amount of an alpha-Amylase peptide or a derivative thereof is administered to a subject in need thereof. In a preferred embodiment the Amylase peptide is pancreatic alpha-Amylase. For example the Amylase peptide is selected from SEQ ID NO 1-11 or a derivative thereof. In another preferred embodiment the Amylase peptide derivative is a composition having at least 90% sequence homology with amino acids 417-427 of SEQ ID NO. 1 and at least 70% sequence homology with the remaining amino acids of SEQ ID NO 1. For example, the method of treating Type I diabetes or Type II diabetes includes one or more of modulating serum insulin, preserving beta-cells, and weight loss. In a preferred embodiment modulating serum insulin includes decreasing insulin levels in the mammalian subject.

Yet another embodiment provides a method for treating obesity in a mammalian subject comprising administering to the subject a therapeutically effective amount of an alpha-Amylase peptide or a derivative thereof. In a preferred embodiment the Amylase peptide is pancreatic alpha-Amylase. For example the Amylase peptide is selected from SEQ ID NO 1-11 or a derivative thereof. In another preferred embodiment the Amylase peptide derivative is a composition having at least 90% sequence homology with amino acids 417-427 of SEQ ID NO. 1 and at least 70% sequence homology with the remaining amino acids of SEQ ID NO 1.

Another embodiment provides for a method of stabilizing serum blood Amylase in a mammalian subject as a method of treating insulin resistance comprising administering to the subject a therapeutically effective amount of an alpha-Amylase peptide or a derivative thereof. In a preferred embodiment the Amylase peptide is pancreatic alpha-Amylase. For example the Amylase peptide is selected from SEQ ID NO 1-11 or a derivative thereof. In another preferred embodiment the Amylase peptide derivative is a composition having at least 90% sequence homology with amino acids 417-427 of SEQ ID NO. 1 and at least 70% sequence homology with the remaining amino acids of SEQ ID NO 1.

Yet another embodiment provides a method of modulating histamine levels in a mammalian subject comprising administering to the subject a therapeutically effective amount of an alpha-amylase peptide or a derivative thereof. In a preferred embodiment the Amylase peptide is pancreatic alpha-Amylase. For example the Amylase peptide is selected from SEQ ID NO 1-11 or a derivative thereof. In another preferred embodiment the Amylase peptide derivative is a composition having at least 90% sequence homology with amino acids 417-427 of SEQ ID NO. 1 and at least 70% sequence homology with the remaining amino acids of SEQ ID NO 1.

Still another embodiment of the present invention provides for a method of treating chronic inflammation in a mammalian subject comprising administering to the subject a therapeutically effective amount of a composition comprising alpha-Amylase. In a preferred embodiment the Amylase peptide is pancreatic alpha-Amylase. For example the Amylase peptide is selected from SEQ ID NO 1-11 or a derivative thereof. In another preferred embodiment the Amylase peptide derivative is a composition having at least 90% sequence homology with amino acids 417-427 of SEQ ID NO. 1 and at least 70% sequence homology with the remaining amino acids of SEQ ID NO 1.

Another embodiment of the present invention provides for a pharmaceutical composition comprising of a peptide selected from SEQ ID NO 1-11 or a derivative thereof.

Yet another embodiment of the present invention provides for a method of treating in a mammalian subject a condition accompanied or caused by IgE mediated histamine release from mast cells comprising administering to a host in need of such treatment a therapeutically effective amount of the pharmaceutical composition according to claim 22. Further administering may be selected from subcutaneous, intramuscular, intraperitoneally, inhalation, intra-arteriole, intravenous, intradermal, topically, oral, perenteral, intraventricular, and intracranial administration. In a preferred embodiment a condition accompanied or caused by IgE mediated histamine release includes allergies and Inflammation, Type I Diabetes, Type II Diabetes, Eczema, Asthma, and Atopic Dermatitis.

Yet another embodiment provides for a skin treatment mixture comprising saline and a peptide selected from SEQ ID NO 1-11 or a derivative thereof.

According to another embodiment of the present invention a compound comprising an alpha-Amylase for use in the treatment of one or more of Type I diabetes, Type II diabetes, Obesity, Insulin resistance, chronic inflammation.

Yet another embodiment of the present invention provides for a compound comprising an alpha Amylase for the treatment of a condition accompanied or caused by IgE mediated histamine release. Such a condition includes allergies, Inflammation, Type I Diabetes, Type II Diabetes, Eczema, Asthma, and Atopic Dermatitis.

One aspect of one embodiment of the present invention provides for a composition which may be an amino acid sequence and/or compound and/or pharmaceutical that is not an insulin analog but rather works to lessen chronic inflammation so that beta-cell function is preserved and secondary complications do not arise as frequently.

Another aspect of one embodiment of the present invention provides for a drug that can act synergistically with current treatments for diabetes (both Type I and Type II) and/or metabolic syndrome X and/or neurologic disorders and/or autism, and/or Alzheimer's.

Another aspect of one embodiment of the present invention provides a pharmaceutical that regulates insulin through the modulation of the proposed metabolic pathway,

Another aspect of one embodiment of the present invention provides for suppression of inflammation through the sequestration of IgE by human pancreatic alpha-Amylase that acts to augment current treatments of CF and extend life expectancy by delaying and or mitigating the severity of respiratory failure and pulmonary disorders.

Another aspect of one embodiment of the present invention provides for the use of Amylase in the treatment of hyperinsulemia.

Another aspect of one embodiment of the present invention provides for the use of Amylase secretion into the blood which can reduce biphasic insulin release via a novel feedback loop.

Another aspect of one embodiment of the present invention provides for the use of Amylase to reduce resistin levels via a mechanism downstream to the inhibition of histamine.

Another aspect of one embodiment of the present invention provides for Amylase to be used in the treatment of allergies.

Another aspect of one embodiment of the present invention provides for Amylase to be used as a natural inhibitor of IgE which in turn inhibits or decreases the release of histamine.

Another aspect of one embodiment of the present invention provides for the use of Amylase in the treatment of insulin resistance.

Another aspect of one embodiment of the present invention provides for stabilizing blood Amylase to treat insulin resistance.

Another aspect of one embodiment of the present invention provides for Amylase is used in the treatment of CVD.

Another aspect of one embodiment of the present invention provides the use of Amylase to inhibit IgE and decrease IgE mediated release from cells, histamine and cortisol.

Another aspect of one embodiment of the present invention provides for modulating cortisol which inhibits the cardioprotective protein p-mTOR via the administration of Amylase to the subject.

Another aspect of one embodiment of the present invention provides for the use of Amylase in the treatment of topical inflammation

Another aspect of one embodiment of the present invention provides for the use of Amylase in the treatment of pre-cancerous and cancerous microenvironments. Pre-cancerous microenvironments are characterized by local inflammation. Amylase's natural anti-inflammatory properties make it a potential treatment for specific types of inflammatory induced cancers such as those associated with metabolic syndrome X.

Another aspect of one embodiment of the present invention provides for the use of Amylase in the treatment of pancreatic tumors and in the treatment of pancreatitis as a pancreatic enzyme supplement.

Another aspect of one embodiment of the present invention provides for the production of human Amylase in yeast which can function as a viable alternative to porcine Amylase. An added benefit of an Amylase from yeast may be reduced risk of allergies.

Another aspect of one embodiment of the present invention provides for the use of Amylase in the treatment of aging such as nephropathy, CVD, and neuropathy.

Another aspect of one embodiment of the present invention provides the use of Amylase for the long term maintenance of serum Amylase levels can improve quality of life in the aging. In fact, Amylase is the only pancreatic enzyme that does not reduce in production during the aging process of healthy individuals.

Another aspect of one embodiment of the present invention provides Amylase as an adjuvant for vaccines.

Another aspect of one embodiment of the present invention provides for the use of Amylase to activate cytokines (IL6, TNF-alpha and IL-B) which increases T cell production and immune response to infection and vaccines.

Another aspect of one embodiment of the present invention provides for the use of Amylase as a treatment for autism. For example, a decrease in Amylase serum concentrations causes downstream effects leading to upregulation of thromboxane and prostaglandin, hormones implicated in autism development.

Another aspect of one embodiment of the present invention provides for the use of Amylase as a treatment for Alzheimer's and other types of brain aging.

Another aspect of one embodiment of the present invention provides for down regulation of Amylase which causes downstream effects leading to decreases in neuronal histamine and decreased leptin which are implicated in disorders such as obesity, age related dementia and neuropathy.

Another aspect of one embodiment of the present invention provides for amino acid residue changes in the Amylase domains/subcomponents to alter Amylase function and binding affinity for IgE. Mutation of the Histidine residue prevents proper Amylase function. Histidine is an essential amino acid whose absence from the Amylase peptide inhibits various functions of the enzyme. We postulate that a histidine deficiency causes not only a malfunctioning Amylase enzyme but also a reduction in the levels of carnosine which play a role in regulating the downstream effectors of Amylase (cytokines). This deficiency can be caused by denaturation by irradiation or loss during first pass metabolism by gut microbes (H. pylori). Therefore, a histidine supplement in addition to Amylase might improve the conditions listed above.

Another aspect of one embodiment of the present invention provides for modulating endotoxins via Amylase. Endotoxins are shown to cause ‘sickness behavior’ and increase leptin production. These can in small quantities be used to treat disorders involving lower leptin levels.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 illustrates a graph from an ELISA showing Amylase inhibits IgE-induced mast cell degranulation and subsequent histamine release.

FIG. 2 illustrates an immunoblot of IgE immunoprecipitated with anti-Amylase antibody and increasing concentrations of BSA-Amylase.

FIG. 3 illustrates an immunoblot of Amylase immunoprecipitated with Anti-IgE antibody;

FIG. 4 is a schematic diagram illustrating an Amylase pathway in an inflammatory process according to one embodiment of the present invention;

FIG. 5 is a schematic diagram illustrating an Amylase/insulin pathway treatable according to one embodiment of the present invention;

FIG. 6 is a schematic diagram illustrating an Amylase/cortisol pathway involved in disease and treatable according to one embodiment of the present invention;

FIG. 7 is a schematic diagram illustrating a diagram of an Amylase/autism pathway involved in disease and treatable according to one embodiment of the present invention;

FIG. 8 is a schematic diagram illustrating high fructose corn syrup impacting an Amylase pathway involved in disease and treatable according to one embodiment of the present invention;

FIG. 9. is a graph of measured serum insulin levels from each group of animals treated;

FIG. 10. is a graph of creatinine vs percentage of total weight loss for each group of animals treated;

FIG. 11 is a graph of protein urea vs creatinine ratio for each group of animals treated.

### DETAILED DESCRIPTION

Alpha-Amylase is an enzyme capable of sequestering IgE, a protein involved in the upregulation of a chronic inflammatory response (for example, the chronic inflammatory response observed in type I diabetes). According to one embodiment of the present invention, disrupting the function of IgE with Amylase provides an ameliorating effect on the chronic inflammation.

The source of chronic inflammation in diabetic patients is tightly linked to the actions of Mast cells. These cells are granule-rich, secretory agents that play a distinct role in the allergic response and inflammation through the release of various inflammatory cytokines and histamine. The degranulation event is triggered by the binding of the previously mentioned IgE molecule to the membrane-bound FceR1 receptor on Mast cells.

Degranulation, in addition to releasing histamine also causes the release of other inflammatory cytokines such as TNF-alpha, IL-1β which cause inflammation and insulin resistance. Stabilization of Mast cells has been shown to prevent the development of both Type I and Type II diabetes and a reduction in inflammatory cytokines helps preserve beta-cell function

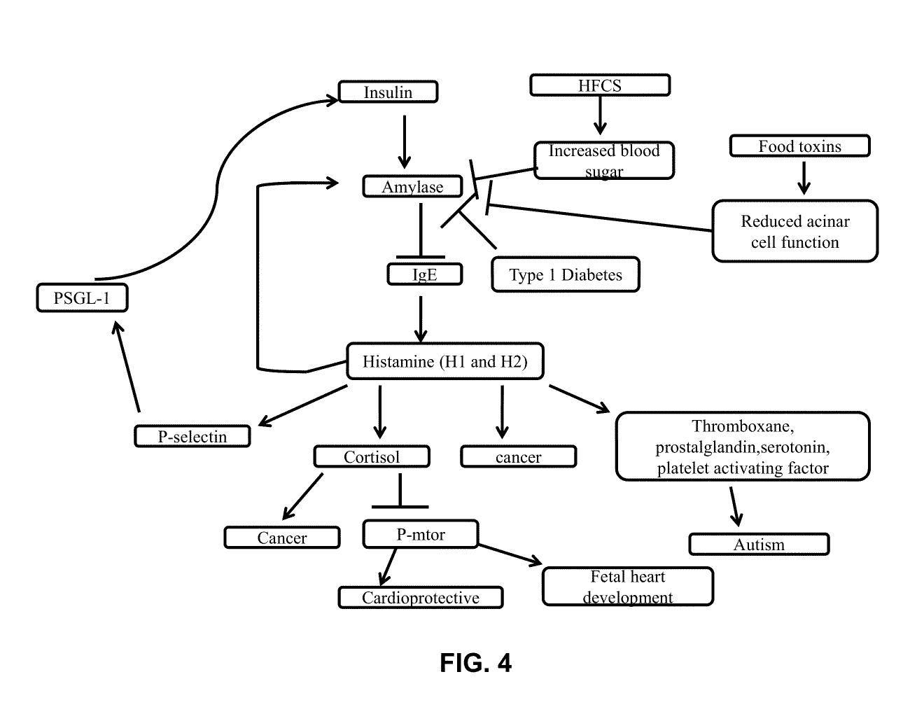

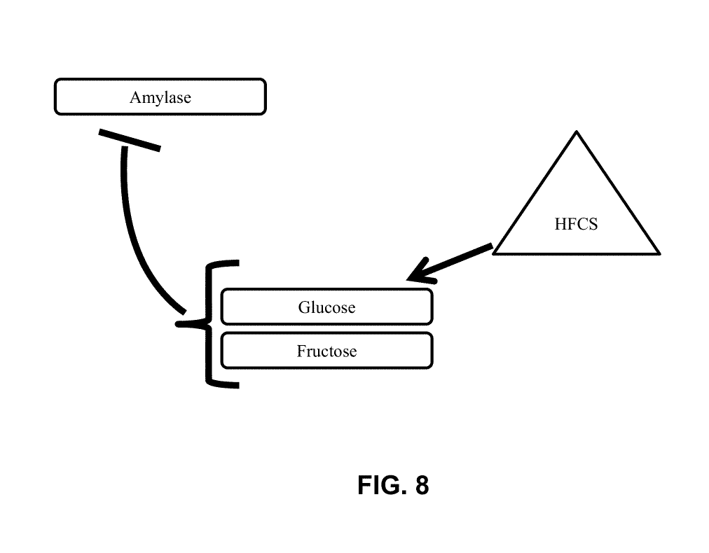

Referring now to FIGS. 4-8, it is postulated that abnormally increased histamine levels are one of the causative factors in the development and progression of Type I diabetes. Histamine activates P-selectin, which in kind binds PSGL-1, leading to insulin release and over time, insulin resistance. Since insulin controls the production of Amylase, this feedback loop exerts substantial control over insulin and Amylase levels. In patients who develop Type I diabetes, this dysfunction in homeostatic regulation leads to insulin resistance.

Histamine also controls the levels of numerous regulatory factors that are implicated in various diseases. One hormone that is upregulated in the presence of histamine is cortisol, a glucocorticoid produced by the adrenal gland. On a systemic level, cortisol is responsible for increasing blood sugar levels and directly counteracts the effects of insulin. It has been shown that excess levels of cortisol can lead to insulin resistance as it acts in opposition to the increased cellular carbohydrate intake triggered by insulin. Corroborating this theory, it has been shown that levels of cortisol are significantly elevated in Type I diabetic patients.

Histamine induces amylase secretion from the pancreas. Excess histamine has also been implicated in increasing resistin levels. Resistin is a pro-inflammatory cytokine that has been identified as having an important role in the pathogenesis of several disorders. In terms of diabetes, studies show a positive correlation between both resistin levels and obesity, and resistin levels and insulin resistance.

Histamine itself is responsible for specific inflammatory responses involved with metabolic disorders such as diabetes mellitus. While inflammation is a natural response to injury and infection, chronic inflammation is thought to disrupt normal cellular activity and damage local tissues. Evolutionary, it is logical to assume the metabolic response and immune response evolved from the same ancestral structures. It is beneficial to have the two responses intimately linked since an immune response should lead to a re-distribution of the body's energy to focus on recovery. However, the balance between the two systems is tightly regulated and exists in a delicate equilibrium. For example, prolonged exposure to pathogens that evoke an inflammatory response has been shown to significantly disrupt normal metabolic processes.

Various studies have shown that Type I diabetics suffer from chronic inflammation. An excess of histamine is physiologically capable of producing this prolonged inflammation and thus disrupting the normal balance between the immune system and the metabolic system. Overall, an agent capable of reducing histamine levels should in theory be able to ameliorate the sustained inflammation observed in diabetic patients. In addition, lowered levels of histamine should control the expression of molecules tightly linked to the development of insulin resistance, such as resistin and cortisol.

The link between Mast cell degranulation and Type I diabetes was discovered by examining the interactions of insulin. Low serum alpha-Amylase levels are associated with insulin deficiency in Type I diabetics (Dandona et al. 1984, Frier et al. 1978, and Swislocki et al. 2005). This was first demonstrated by evidence from several animal models exhibiting a correlation between reduced levels of pancreatic Amylase and development of insulin resistance (Schneeman et al. 1983 and Trimble et al. 1986). A more recent study has shown that serum Amylase levels are inversely related with cardiovascular/metabolic risk factors and that low serum Amylase levels precede metabolic dysfunction (Nakajima et al. 2011a).

Insulin itself is tightly linked to Amylase as the hormone regulates the expression of the pancreatic Amylase gene AMY2 by acting as a transcription factor (Boulet et al. 1986, Johnson et al. 1993, Soling et al. 1972). Thus, the insulin deficiency found in diabetes leads to lowered levels of serum Amylase. This releases the inhibitory effect of Amylase on IgE and greatly increases the rate of Mast cell degranulation and subsequent levels of inflammation. In addition, elevated levels of IgE have alone been implicated in the promotion of inflammation through the increased expression of IFN-γ and IL-6 cytokines (Sun et al. 2007 and Wang et al. 2011).

A previously unknown interaction between the pancreatic alpha-Amylase peptide and IgE is described herein. Through our in vitro work and computation modeling, we have demonstrated the ability of alpha Amylase to bind and sequester the IgE antibody. Modulation of the interaction is a target for therapeutic intervention. For example, a compound according to one embodiment of the present invention may be used to lower rates of Mast cell degranulation within the body. Mast cells, which have been highly characterized for their role in allergies, are also powerful effectors of the inflammatory response. They contain many different inflammatory molecules such as histamine, cytokines, and interleukins, which are all released upon degranulation. The mode of Amylase action (see for example FIGS. 4-9) can be modulated according to an embodiment of the present invention. Methods of treatment according to one embodiment of the present invention differ from the current experimental anti-inflammatory treatments as compounds disclosed herein act mechanistically upstream of those that target the CD3 receptor (Damle and Doyle, 1989).

Sequence Alignment of the Alpha Amylase Protein (homo sapiens)

Sequence 1B corresponds to SEQ ID NO 3, sequence 1C corresponds to SEQ ID NO 4, sequence 1A corresponds to SEQ ID NO 5, sequence 2A corresponds to SEQ ID NO 1 and sequence 2B corresponds to SEQ ID NO 2.

```
1B MKLFWLLFTIGFCWAQYSSNTQQGRTSIVHLFEWRWVDIALECERYLAPKGFGGVQVSPP 60
1C MKLFWLLFTIGFCWAQYSSNTQQGRTSIVHLFEWRWVDIALECERYLAPKGFGGVQVSPP 60
1A MKLFWLLFTIGFCWAQYSSNTQQGRTSIVHLFEWRWVDIALECERYLAPKGFGGVQVSPP 60
2A MKFFLLLFTIGFCWAQYSPNTQQGRTSIVHLFEWRWVDIALECERYLAPKGFGGVQVSPP 60
2B MKFFLLLFTIGFCWAQYSPNTQQGRTSIVHLFEWRWVDIALECERYLAPKGFGGVQVSPP 60
      **:* *************.*****************************************
1B NENVAIHNPFRPWWERYQPVSYKLCTRSGNEDEFRNMVTRCNNVGVRIYVDAVINHMCGN 120
1C NENVAIHNPFRPWWERYQPVSYKLCTRSGNEDEFRNMVTRCNNVGVRIYVDAVINHMCGN 120
lA NENVAIHNPFRPWWERYQPVSYKLCTRSGNEDEFRNMVTRCNNVGVRIYVDAVINHMCGN 120
2A NENVAIYNPFRPWWERYQPVSYKLCTRSGNEDEFRNMVTRCNNVGVRIYVDAVINHMCGN 120
2B NENVAIHNPFRPWWERYQPVSYKLCTRSGNEDEFRNMVTRCNNVGVRIYVDAVINHMSGN 120
      ******:**************************************************.**
1B AVSAGTSSTCGSYFNPGSRDFPAVPYSGWDFNDGKCKTGSGDIENYNDATQVRDCRLSGL 180
1C AVSAGTSSTCGSYFNPGSRDFPAVPYSGWDFNDGKCKTGSGDIENYNDATQVRDCRLSGL 180
1A AVSAGTSSTCGSYFNPGSRDFPAVPYSGWDFNDGKCKTGSGDIENYNDATQVRDCRLSGL 180
2A AVSAGTSSTCGSYFNPGSRDFPAVPYSGWDFNDGKCKTGSGDIENYNDATQVRDCRLTGL 180
2B AVSAGTSSTCGSYFNPGSRBFPAVPYSGWDFNDGKCKTGSGDIENYNDATQVRDCRLVGL 180
      ********************************************************* **
1B LDLALGKDYVRSKIAEYMNHLIDIGVAGFRIDASKHMWPGDIKAILDKLHNLNSNWFPEG 240
1C LDLALGKDYVRSKIAEYMNHLIDIGVAGFRIDASKHMWPGDIKAILDKLHNLNSNWFPEG 240
1A LDLALGKDYVRSKIAEYMNHLIDIGVAGFRIDASKHMWPGDIKAILDKLHNLNSNWFPEG 240
2A LDLALEKDYVRSKIAEYMNHLIDIGVAGFRLDASKHMWPGDIKAILDKLHNLNSNWFPAG 240
2B LDLALEKDYVRSKIAEYMNHLIDIGVAGFRLDASKHMWPGDIKAILDKLHNLNSNWFPAG 240
      ***** ************************:*************************** *
1B SKPFIYQEVIDLGGEPIKSSDYFGNGRVTEFKYGAKLGTVIRKWNGEKMSYLKNWGEGWG 300
1C SKPFIYQEVIDLGGEPIKSSDYFGNGRVTEFKYGAKLGTVIRKWNGEKMSYLKNWGEGWG 300
1A SKPFIYQEVIDLGGEPIKSSDYFGNGRVTEFKYGAKLGTVIRKWNGEKMSYLKNWGEGWG 300
2A SKPFIYQEVIDLGGEPIKSSDYFGNGRVTEFKYGAKLGTVIRKWNGEKMSYLKNWGEGWG 300
2B SKPFIYQEVIDLGGEPIKSSDYFGNGRVTEFKYGAKLGTVIRKWNGEKMSYLKNWGEGWG 300
      ************************************************************
1B FMPSDRALVFVDNHDNQRGHGAGGASILTFWDARLYKMAVGFMLAHPYGFTRVMSSYRWP 360
1C FMPSDRALVFVDNHDNQRGHGAGGASILTFWDARLYKMAVGFMLAHPYGFTRVMSSYRWP 360
1A FMPSDRALVFVDNHDNQRGHGAGGASILTFWDARLYKMAVGFMLAHPYGFTRVMSSYRWP 360
2A FVPSDRALVFVDNHDNQRGHGAGGASILTFWDARLYKMAVGFMLAHPYGFTRVMSSYRWP 360
2B FMPSDRALVFVDNHDNQRGHGAGGASILTFWDARLYKMAVGFMLAHPYGFTRVMSSYRWP 360
      *:**********************************************************
1B RYFENGKDVNDWVGPPNDNGVTKEVTINPDTTCGNDWVCEHRWRQIRNMVNFRNVVDGQP 420
1C RYFENGKDVNDWVGPPNDNGVTKEVTINPDTTCGNDWVCEHRWRQIRNMVNFRNVVDGQP 420
1A RYFENGKDVNDWVGPPNDNGVTKEVTINPDTTCGNDWVCEHRWRQIRNMVNFRNVVDGQP 420
2A RQFQNGNDVNDWVGPPNNNGVIKEVTINPDTTCGNDWVCEHRWRQIRNMVIFRNVVDGQP 420
2B RQFQNGNDVNDWVGPPNNNGVIKEVTINPDTTCGNDWVCEHRWRQIRNMVNFRNVVDGQP 420
      * *:**:**********:*** **************************** *********
1B FTNWYDNGSNQVAFGRGNRGFIVFNNDDWTFSLTLQTGLPAGTYCDVISGDKINGNCTGI 480
1C FTNWYDNGSNQVAFGRGNRGFIVFNNDDWTFSLTLQTGLPAGTYCDVISGDKINGNCTGI 480
1A FTNWYDNGSNQVAFGRGNRGFIVFNNDDWITSLTLQTGLPAGTYCDVISGDKINGNCTGI 480
2A FTNWYDNGSNQVAFGRGNRGFIVFNNDDWSFSLTLQTGLPAGTYCDVISGDKINGNCTGI 480
2B FTNWYDNGSNQVAFGRGNRGFIVFNNDDWTFSLTLQTGLPAGTYCDVISGDKINGNCTGI 480
      *****************************:******************************
1B KIYVSDDGKAHFSISNSAEDPFIAIHAESKL 511
1C KIYVSDDGKAHFSISNSAEDPFIAIHAESKL 511
lA KIYVSDDGKAHFSISNSAEDPFIAIHAESKL 511
2A KIYVSDDGKAHFSISNSAEDPFIAIHAESKL 511
2B KIYVSDDGKAHFSISNSAEDPFIAIHAESKL 511
      *******************************
```

The isoform sequence alignment of alpha Amylase (1A, 1B, 1C, 2A, and 2B) was prepared using ClustalW. Isoform Sequence Alignment of alpha-Amylase. Accession numbers of the alpha-Amylase sequences used in the protein sequence alignment are: NP_{—}004029—salivary Amylase alpha 1A precursor; NP_{—}001008219—salivary Amylase alpha 1B precursor; NP_{—}001008220—salivary Amylase alpha 1C precursor; NP_{—}000690—pancreatic Amylase alpha 2A precursor; NP_{—}066188—pancreatic Amylase alpha 2B precursor. The sequences were further analyzed with protParam to identify the following: Number of amino acids: 511; Molecular weight: 57767.8; Theoretical pI: 6.47; Total number of negatively charged residues (Asp+Glu): 55; Total number of positively charged residues (Arg+Lys): 52 Atomic composition: Carbon C 2589 Hydrogen H 3857 Nitrogen N 715 Oxygen O 752 Sulfur S 23 Formula: C₂₅₈₉H₃₈₅₇N₇₁₅O₇₅₂S₂₃; Total number of atoms: 7936; Extinction coefficients: Extinction coefficients are in units of M⁻¹ cm⁻¹, at 280 nm measured in water. Ext. coefficient 136540 Abs 0.1%(=1 g/l) 2,364, assuming ALL Cys residues appear as half cystines Ext. coefficient 135790 Abs 0.1%(=1 g/l) 2.351, assuming NO Cys residues appear as half cystines. Estimated half-life: The N-terminal of the sequence considered is M (Met). The estimated half-life is: 30 hours (mammalian reticulocytes, in vitro). >20 hours (yeast, in vivo). >10 hours (Escherichia coli, in vivo). Instability index: The instability index (II) is computed to be 23.58 This classifies the protein as stable. Aliphatic index: 67.12. Grand average of hydropathicity (GRAVY): −0.436. The sequence is presented using abbreviations for amino acids.

In Silico

Active Site Identification:

BLAST analysis of human pancreatic Amylase with the known sequence of IgE receptor domain binding sites shows some regions of homology. Specifically, the homologous region is located between residues 417-427. Structural analysis has also revealed that analogous residues in human Amylase and the IgE receptor domain form internal pockets within the protein and are not surface-exposed. These pockets are characteristic of those typically found in binding sites.

Structural Peptide Analysis:

The primary amino acid sequences of both the porcine and the human Amylase were examined using in silico methods (Swiss-Prot accession codes P00690 and P04746). While both the porcine and human Amylase show homology in key regions, there are noticeable and significant differences between the two. The program PONDr was run on both sequences to determine-the regions of disorder in both proteins. Disordered regions are characterized by a lack of tertiary structure. The results show that the human and porcine Amylase exhibit different regions of disorder. There are various metrics to predict disorder from a protein sequence. Analysis was done using the VLXT and the Can_XT metrics.

The VLXT predictor integrates three feedforward neural networks: the VL1 predictor (Romero et al. 1997), the N-terminus predictor (XN), and the C-terminus predictor (XC) (both from Li et al. 1999). A simple average is taken for the overlapping predictions. A sliding window of nine amino acids is used to smooth the prediction values along the length of the sequence. The CaN predictor is a feedforward neural network that was trained on regions of 13 calcineurin proteins that were identified by sequence homology with the known disordered region of human calcineurin (Romero et al., 1997).

According to both metrics, there were significantly greater regions of disorder between the human and porcine Amylase peptides. VLXT predicted five regions of disorder in the porcine Amylase while only three regions in the human peptide. Similarly, Can_{‘3}XT predicted four regions of disorder in the porcine Amylase and three regions in the human Amylase. The difference in structural stability between the human and porcine Amylases implies altered functionality of the peptide. There is also a variance in glycosylation sites between the human and porcine Amylases. The porcine Amylase is glycosylated at residue 426 and this residue is not exposed to the surface. However, the human Amylase is glycosylated on a surface residue (476) which allows the sugar to potentially modulate cellular signaling and adhesion. The unexposed porcine sugar is unable to perform these same activities, leading to another potential difference in function between the variant species.

Analysis of the human and porcine show that the two have different post-translational modifications. Specifically, the Swiss-Prot database shows a phosphorylation site for human Amylase which is not present in the porcine Amylase. The apparent discrepancy between the glycosylation and phosphorylation sites between human and porcine Amylases further suggests that the secondary function of the two Amylases is dissimilar and that human Amylase has some unique functions.

Active Site Identification:

The Basic Local Alignment Search Tool (BLAST) finds regions of local similarity between sequences. The program used compared protein sequences to sequence databases and calculates the statistical significance of matches. BLAST can be used to infer functional and evolutionary relationships between sequences as well as help identify members of gene families. BLAST analysis of human and porcine Amylase with the known sequence of IgE receptor domain binding sites show homology between porcine and human Amylases. Specifically, the homologous region is located between residues 417-427 WDGQPFTNWYDNGSN with the underlined amino acids being conserved.

The porcine Amylase is glycosylated at residue 426 while neither the human Amylase nor the IgE receptor are glycosylated. This implies greater structural similarity between the human Amylase and IgE receptor since the presence of the sugar on the porcine Amylase would negatively impact binding with the IgE molecule. Structural analysis showed that analogous residues in human Amylase and the IgE receptor domain form internal pockets within the protein and are not surface-exposed. These pockets are typical characteristics of binding sites.

According to one embodiment a composition comprising amino acids 1-511 of the pancreatic alpha-Amylase peptide or a derivative thereof wherein the derivative maintains 70% homology to the peptide sequence outside of the active site and 90% homology within the peptide active site. In a preferred embodiment the peptide is from human pancreatic alpha Amylase. Another embodiment provides for a composition comprising a peptide selected from SEQ ID NO 1-11 or a derivative thereof wherein the derivative maintains at least 70% homology to the peptide sequence outside of the active site and at least 90% homology within the peptide active site. In yet another embodiment a composition comprising SEQ ID NO 6 is used to treat subjects presenting with the conditions disclosed herein or to modulate IgE mediated histamine release. In an alternative embodiment the derivative maintains at least 80% homology to the peptide sequence outside of the active site and at least 90% homology within the peptide active site or in a more preferred embodiment the derivative maintains at least 90% homology to the peptide sequence outside of the active site and at least 90% homology within the peptide active site. In another embodiment the peptides and derivatives described herein also include the pharmaceutically acceptable salts thereof.

In Vitro

ELISA:

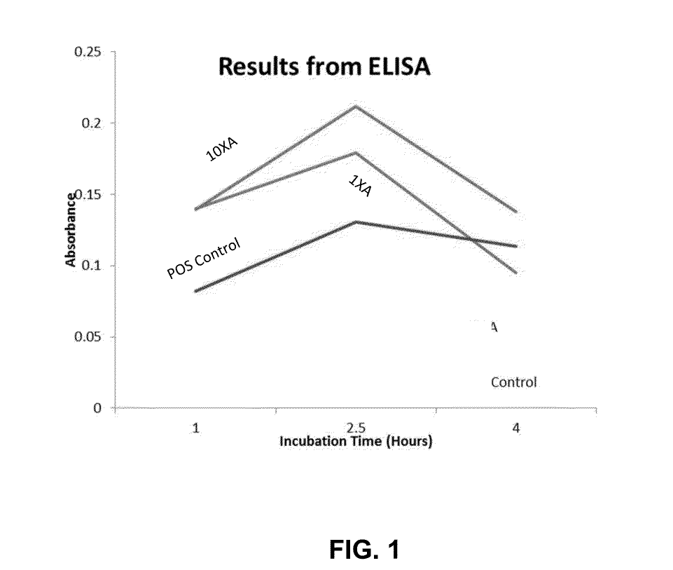

Referring now to FIG. 1, Mast cells were treated with IgE, anti-IgE and varying doses of alpha-Amylase in a medium that would permit binding. In this experiment, Mast cells were grown to confluence, harvested and resuspended in 8 ml PBS and a 3 ml aliquot was plated in triplicate. 0.29 ml (100 Units) of Amylase was added to 0.7 mL PBS bringing the total volume to 1 mL. 3 μL was used in the 1× Amylase dose and 30 μL was used in the 10× Amylase dose. 1 set of triplicates was incubated with 1× Amylase (100 Units/L) and the second set with 10× Amylase (1000 Units/L). After 10 minutes, 250 μL of IgE was added, and then 200 μL of anti-IgE. The cells were incubated at 37 degrees Celsius during this time. 50 μL was taken at 1 hour, 2.5 hours and 4 hours and subjected to histamine level analysis via ELISA. A control set was co-incubated with PBS.

Anti-IgE when added to IgE-incubated Mast cells causes degranulation. Histamine release occurs during degranulation. Measurements were taken from all three subsets at 1 hour, 2.5 hours and 4 hours and subjected to analysis of histamine levels by ELISA from Oxford Biolabs. FIG. 1 is a graph of the results of an ELISA in which histamine levels are measured. Higher absorbance rates correlate to lower levels of Histamine. At 1 hour, the levels of Histamine released in the 1× and 10× doses were the same, with higher release in the negative control. At 2.5 hours there is dose dependency with less histamine release compared to 1 hour (due to the degradation of Histamine). At 4 hours, there is greater histamine release in the 1× and 10× doses implying that the sequestration of IgE by Amylase is not permanent.

Degranulation based upon the Immunoglobulin-E (IgE)-Anti-IgE reaction is well documented in the literature. Histamine levels were detected by an ELISA kit and served as a marker for degranulation. Histamine levels were lower in a manner that was both time and dose dependent in the samples treated with Amylase. This implies that cellular degranulation was prevented in a time and dose-dependent manner by the addition of Amylase. These results suggest that Amylase inhibits IgE-induced Mast cell degranulation and subsequent histamine release.

Our studies indicate that Amylase directly binds to IgE in a key binding site. Physiologically, this binding event leads to an inhibition of the binding of IgE to the IgE receptor on Mast cells. Normally, the binding event of IgE to its receptor is triggered by the presence of an antigen and leads to degranulation of the mast cell and histamine release. Due to the presence of Amylase, IgE binding to its receptor is inhibited and prevents degranulation and histamine release. The inhibition of histamine has several physiological implications discussed herein.

Preliminary results show that Amylase directly binds to IgE. Physiologically, this binding event leads to an inhibition of the antibody's normal binding to the FceR1 receptor on the surface of Mast cells. Normally, the binding of IgE to its receptor, in the presence of the proper antigen, leads to degranulation of the mast cell and histamine release. Due to the sequestering effect exhibited by Amylase, IgE is incapable of binding and triggering mast cell degranulation.

Co-Immunoprecipitation:

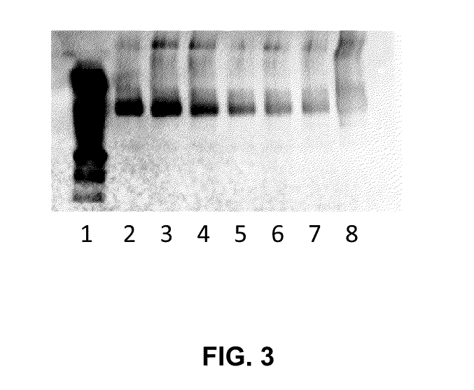

Referring now to FIG. 3, an immunoblot is shown having samples in lanes 1-8. The samples are prepared as follows. 10 μg of Amylase was mixed with 1 μg of IgE and incubated overnight at 4° C. and then for one hour at 37° C. Anti-Amylase antibody was used for immunoprecipitation. Anti-IgE antibody was used for immunoblotting. BSA was used as a competitive binding control. A sample with no IgE and 0.5 mg of BSA+10 microgram of Amylase (lane 2) was used as negative control for immunoblotting. (Lanes 3 and 4 represent 0.5 μg BSA, lanes 5 and 6 represent 0.5 μg BSA and lanes 7 and 8 represent 0.05 μg BSA). After immunoprecipitation, pellets were dissolved in 45 microliters of Laemmli sample buffer and 15 microliters were loaded on the gel. 1 microgram of IgE was used as positive control (lane 1). Results showed that IgE immunoprecipitated with Amylase under conditions of increasing concentrations of BSA, demonstrating little non-specific binding.

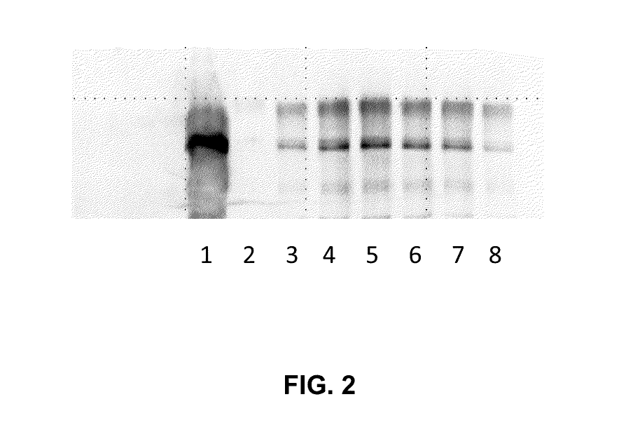

Referring now to FIG. 2, an immunoblot is shown having samples in lanes 1-8. The samples are prepared as follows. 1 μg of IgE was mixed with Amylase at varying concentrations 0.5 μg (lanes 7 and 8), 5 μg (lanes 5 and 6) and 10 μg (lanes 3 and 4 and incubated for overnight at 4° C. and then one hour at 37° C. Anti-IgE antibody was used for immunoprecipitation. Anti-Amylase antibody was used for immunoblotting. A sample with no Amylase and 1 μg of IgE (lane 2) was used as negative control for immunoblotting. 5 micrograms of Amylase was used as the positive control (lane 1). Results showed the binding of Amylase and IgE was observed to behave in a dose-dependent manner. Based upon FIGS. 2 and 3, The IgE-Amylase interaction is specific and dose-dependent

A number of characteristics from initial in silica and in vitro studies indicate that Amylase plays a role in the inhibition of peptides upregulated in chronic inflammation. Specifically, it has been shown to stabilize Mast cells and decrease the release of inflammatory cytokines and interleukins from those cells. These inflammatory molecules increase the autoimmune response that causes beta-cell death and are directly responsible for the development of CVD, kidney disease, nerve damage, eye damage and osteoporosis. These disorders are all common in Type I diabetics.

In silica modeling identified a sequence of residues present in Amylase that shares both sequence homology and structural homology with an Fc Receptor IgE-binding domain. In vivo studies in Mast cells show that Amylase prevents degranulation of the cells when treated with IgE and its antibody in both a dose dependent and time dependent manner. Co-Immunoprecipitation studies have confirmed binding interactions between Amylase and IgE. We hypothesize that it is this binding and sequestering of IgE that prevents mast cell degranulation.

Researchers have been looking into the use of alpha-Amylase inhibitors as a way to lower blood glucose levels in diabetics (Kumar et al. 2011). Alpha-Amylase is the first step in digestion of oligosaccharides into glucose. Glycosidase is the second. Inhibiting digestion of oligosaccharides will presumably slow and prevent the formation of glucose in your blood stream. There are several drugs on the market that function in this manner and are currently in use for Type II diabetic patients. Acarbose (Trade name: Precose) and miglitol (Trade name: Glyset) function as competitive, reversible inhibitors of alpha glucosidase and alpha-Amylase (Koski R R 2006). Several herbal remedies have been investigated as well and are thought to function in very similar ways by inhibiting alpha-Amylase (Ali et al. 2006, Kim et al. 2000 and Subramanian et al. 2008). Other researchers have investigated the use of Amylase inhibitors in animals. Koike et al. (1994) observed that using Amylase inhibitors (wheat Amylase inhibitor) lowered blood glucose levels without affecting pancreatic growth and might be able treat diabetes but were ultimately unsure of whether the dose could also treat obesity. Similarly, Kataoka et al. (1999) used wheat Amylase inhibitor in rats and observed similar results to Koike et al.

According to one embodiment of the present invention an Amylase supplement may be used to treat hyperinsulinemia, both Type II and I diabetes, metabolic syndrome, and other various metabolic disorders of glucose and insulin. In a preferred embodiment the Amylase supplement comprises amino acids 417-427 of the human pancreatic alpha-Amylase amino acid sequence. In another embodiment the full length peptide is utilized.

### Experiments

### Animal Trials

BKS.Cg-Dock7m mice were treated with control and an Amylase compound according to an embodiment of the present invention. In the absence of treatment, the BKS.Cg-Dock7m mice are homozygous for the diabetes spontaneous mutation (Lepr^{db}) and become obese at approximately three to four weeks of age. The mice begin to exhibit hyperinsulemia at 2 weeks and do not survive for longer than 10 months. At the beginning of the animal trial, the test animals were obese, severely hyperinsulemic and hyperglycemic. After 28 consecutive days of dosing, the animals were euthanized and tissue analyzed. Compounds described herein were tested against the positive control of Metformin hydrochloride (“Metformin”)

### Administration: Oral Gavage

Animals were manually restrained and administered Metformin by oral gavage using a disposable gavage needle. The target dose was 5 mL/kg, and actual dose volumes ranged from 190 to 260 μL/animal.

Test Article Administration: Intraperitoneal (IP) Injection

### 1× Dilution:

Amylase was purchased from Abcam (product ID: ab77861) at a dilution of 340 Units/mL. Each animal was treated with Amylase to achieve a dilution of 80 Units/L of blood. The test compound was diluted in phosphate buffer saline (PBS) and each day the test animals were injected with 300 uL of the Amylase-PBS solution. For every 299.4 uL of PBS, 0.6 uL of Amylase was used to achieve a concentration of 80 Units/Liter assuming a body weight of 45 g. The aliquots were stored at −80 C.

### 3× Dilution:

Amylase was purchased from Abcam (product ID: ab77861) at a dilution of 340 Units/mL. Each animal was treated with Amylase to achieve a dilution of 240 Units/L of blood. The test compound was diluted in phosphate buffer saline (PBS) and each day the test animals were injected with 300 uL of the Amylase-PBS solution. For every 298.3 uL of PBS, 1.7 uL of Amylase was used to achieve a concentration of 80 Units/L assuming a body weight of 45 g. The aliquots were stored at −80 degrees C.

1× (used synonymously with TV1) and 3× (used synonymously with TV2) doses of human alpha-Amylase enzyme (referred to herein as Test Compound 1) were determined to be 80 Units/Liter of blood volume and 240 Units/Liter of blood volume respectively. Animals were manually restrained and administered a target volume of 300 μL of phosphate buffered saline (PBS) (vehicle), by IP injection, once per day, on Days 1-28. Actual dose volumes changed throughout the study and ranged from 300 to 310 μL. Endpoints include daily clinical observations and body weights. Creatinine, insulin, Protein Urea/Creatinine levels, clinical pathology and gross pathology at necropsy were also obtained. The mice were euthanized and necropsied on Day 28.

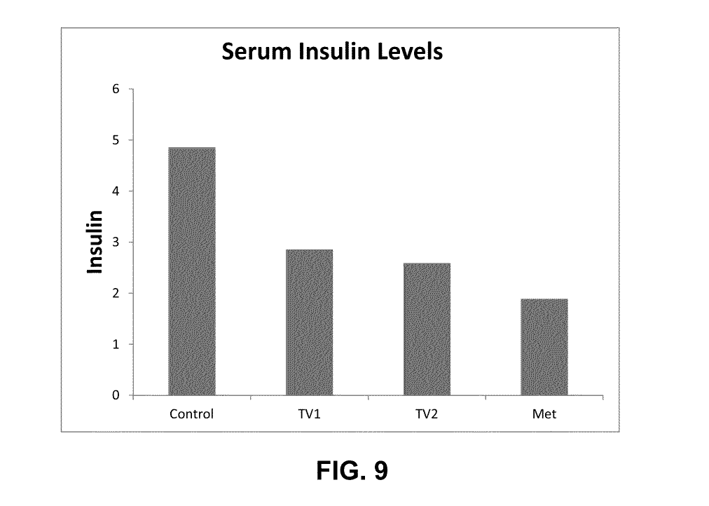

Referring now to FIG. 9, insulin values were measured in serum from four animals from each treatment group at the time of necropsy following 28 consecutive days of dosing with Control (metformin), Test Compound 1 (SEQ ID NO 1), or vehicle alone. Animals in treatment group 1 received negative control (vehicle). Animals in treatment group 2 received 1× dose (80 Units/liter of blood volume) of Test Compound 1 (also known as TV1 herein). Animals in treatment group 3 received 3× dose (240 Units/liter of blood volume) of Test Compound 1 and (referenced herein as TV2). Animals in treatment group 4 received metformin as a control. Serum insulin was assayed using mouse/rat specific insulin ELISA kits from Millipore (Cat #EZRMI-13K), according to the manufacturer's instructions. The processed plates were read using a Molecular Devices VersaMax plate reader.

Animals treated with Test Compound 1 at either 1× or 3× doses and animals treated with Metformin appeared to have moderate decreases in serum insulin levels compared to vehicle control animals. An optimal insulin level is 1. Animals from Treatment Group 3 did exhibit a change in insulin levels.

Body weights were measured prior to the study for randomization. Additional weekly body weights were collected throughout the study. We also were interested in weight loss for the animals for various treatment regimens. Referring now to Table 1 weight loss data as a percent weight loss with the reference being the weight before any treatment is illustrated for each test group. There is a dose-dependency in weight loss with our 1× of Test Compound 1 and 3× of Test Compound 1 doses outperforming metformin. As with the insulin data, there is a dose dependent trend between 1× and 3×. The higher dose of Test Compound 1 outperforms metformin.

TABLE 1
Body Weight % Lost

| Test Vehicle | Neg Control | 1X | 3X | Metformin |
|---|---|---|---|---|
| % Weight loss raw | 6% | 5.5% | 7.4% | 7.4% |
| % Weight loss n = 6 | 4.6% | 5.2% | 8.3% | 6.9% |
| % Weight loss n = 4 | 4.5% | 5% | 10% | 7% |

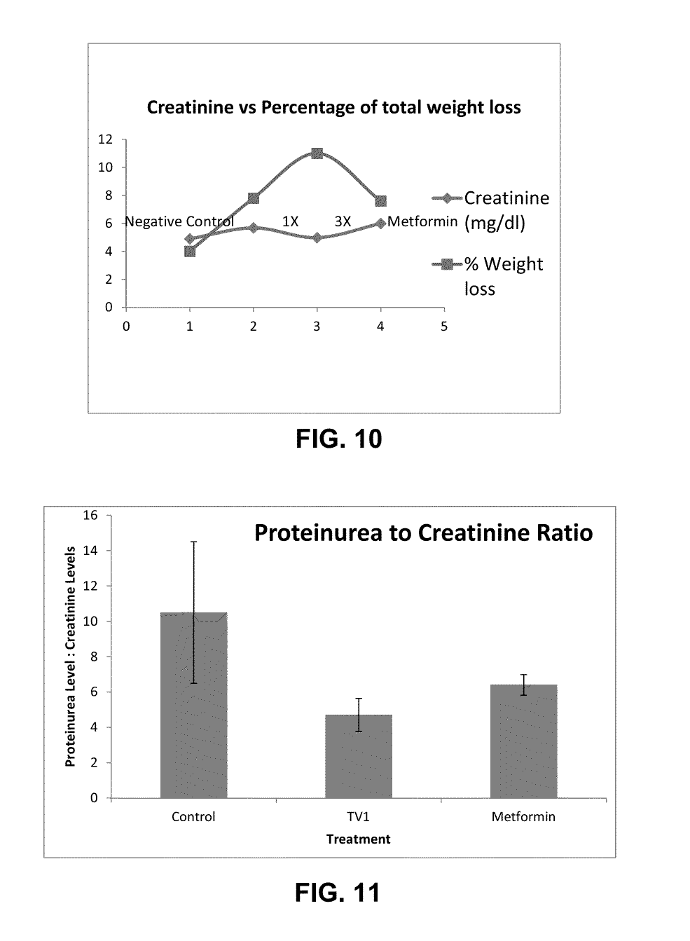

The source of the weight loss, (whether fat or muscle) is physiologically critical. Ideally, the majority of weight lost would be fat and not muscle. Referring now to FIG. 10, to quantify the source of the weight loss, we examined urine creatinine levels in the test animals. If the muscle-fat weight loss ratio was equal in all samples, weight loss and creatinine levels would be inversely related. The creatinine data does not show this relationship. The data in FIG. 10 indicates that in the 1× dose of Test Compound 1 group, the majority of weight lost is fat content and muscle mass is preserved. This also implies that the test animals treated with 1× dose of Test Compound 1 retained more muscle mass compared to the controls.

Referring now to FIG. 11, Protein Urea/Creatinine Levels are measured and shown. Creatinine levels are directly related to muscle mass in mice and so higher Creatinine is correlated with higher muscle mass. A healthy Protein Urea to Creatinine level is 1. Higher levels imply kidney damage. Urine measured from the animals in treatment group 2 demonstrated a lower Protein Urea to Creatinine ratio compared to urine from animals in treatment group 4 or animals in treatment group 1 indicating that Test Compound 1 like metformin control is nephroprotective. Optimal Protein Urea: Creatinine is 1.

The composition, method of using the composition and method of treatment described herein is non-obvious as it is literally the opposite of the current accepted theories in the field. Keeping pancreatic Amylase levels in the blood at a consistent, healthy level within a standard range can treat and aid the disorders and diseases mentioned above. The standard range may be determined based upon the standard range for an individual or may be within a range identified by others for example O'Donnell MD, Fitzgerald O, McGeeney K F, (1977). Differential Serum Amylase Determination by Use of an Inhibitor, and Design of a Routine Procedure. Clinical Chemistry 23: 560-566; Pancreatic Amylase Levels (Male)—A mean of 82.4 units per liter with a standard deviation of 29.9 units; Pancreatic Amylase Levels (Female)—A mean of 99.5 units per liter with a standard deviation of 29.8 units. Any values lower than two standard deviations from the mean will be considered to be a treatable Amylase deficiency.

This is due to Amylase's anti-inflammatory properties. By treating an individual with human Amylase it is possible to restore proper functioning of the insulin-secretion pathway. The development and production of human-grade Amylase, currently unavailable as a therapeutic will be useful as a method of treating hyperinsulinemia.

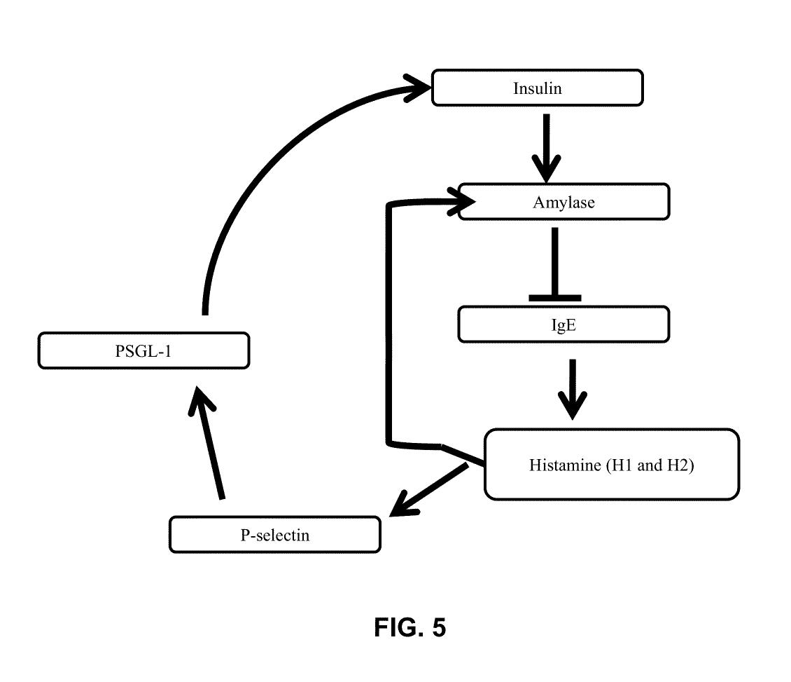

Histamine is a nitrogen containing compound that is critical for various physiological processes including the immune response. Referring now to FIG. 5, histamine controls the level of several key proteins including P-selectin. Higher histamine levels lead to higher levels of P-selectin. P-selectin binds to and directly activates P-Selectin Glycoprotein (PSGL-1). PSGL-1 is a mucin-like adhesion protein that is expressed on the surfaces of cells. Animal studies conducted by Sato et AI. showed that PSGL-1 is positively correlated to insulin resistance. PSGL-1 activation directly leads to insulin secretion. PSGL-1 inactivation prevents insulin secretion. Specifically, DNA microarray analysis showed that adipose tissue of db/db (leptin deficient) mice showed significantly increased levels of PSGL-1. The study further showed that increased PSGL-1 activity leads to the release of insulin and causes insulin resistance and inflammation. Insulin is a direct transcription factor for Amylase and therefore increased levels of insulin will lead to higher levels of Amylase. To summarize, Amylase reduces histamine release by binding to IgE. Histamine activates P-Selectin which in turn produces active PSGL-1 which leads to insulin release and insulin resistance. Since insulin controls the production of Amylase, this feedback loop keeps tight control over insulin and Amylase levels. In patients who are Type II Diabetic, this feedback loop is dysfunctional and eventually leads to insulin resistance. The presence of Amylase or an Amylase derivative composition will help regulate insulin levels and prevent insulin resistance. Amylase inhibition increases biphasic insulin release as well as the histamine response causing insulin resistance and allergies.

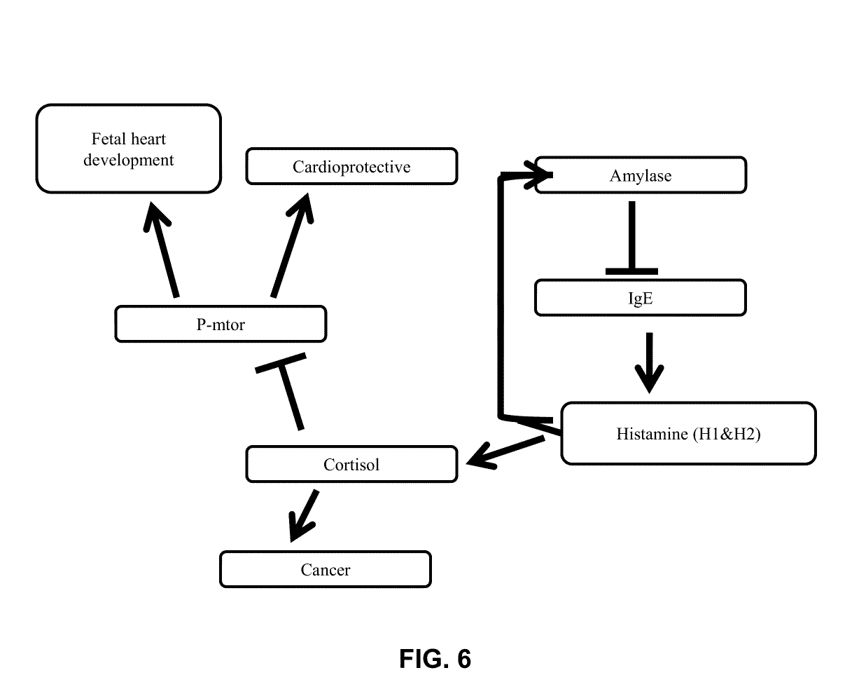

Histamine also controls the levels of various other regulatory factors that are implicated in various disease states. Referring now to FIG. 6, one such hormone that is upregulated in the presence of histamine is cortisol. Cortisol is a glucocorticoid and is produced by the adrenal gland. Overall, cortisol is responsible for increasing blood sugar levels and therefore, in essence, counteracts the effects of insulin. It has been shown that excess levels of cortisol can lead to insulin resistance since cortisol directly prevents insulin from performing its natural function of facilitating transport of sugar from the blood into cells. Cortisol is also implicated in the etiology of many e cancers, and inhibits P-mTOR which in part controls fetal heart development. P-mTOR is cardioprotective. Furthermore, it has been shown that the levels of cortisol are significantly elevated in Type II diabetic patients which further substantiates the key role of cortisol in diabetes. Histamine increases the levels of cortisol in patients. In cases where there is too much histamine, this clearly can contribute to the progression of insulin resistance and ultimately diabetes. Since Amylase is able to inhibit the synthesis of histamine, this could help control cortisol levels and ultimately help counteract insulin resistance.

Excess histamine has also been implicated in increasing resistin levels. Resistin is a pro-inflammatory cytokine that is a key player in inflammatory diseases. Studies shows that resistin levels and obesity are highly correlated and this could provide reason for the increase in inflammatory diseases in obese patients. Additionally, studies show that insulin resistance and serum resistin levels are positively correlated. Since histamine is a key driving force for the upregulation of resistin, Amylase, which inhibits the release of histamine, can help reduce resistin levels.

Histamine itself is responsible for inflammatory responses and these responses have been directly connected with metabolic disorders such as Type II diabetes. While this seems a bit counterintuitive since inflammation response is critical for tissue repair, the long term consequences of such inflammation are quite deleterious. Evolutionary, the metabolic response and immune response evolved from the same ancestral structures. Furthermore, it is beneficial to have the two responses intimately linked since an immune response should lead to a re-distribution of the body's energy to focus on the inflamed area. However, this balance between metabolic response and immune response is delicate and over-exposure to either of the two can disturb this balance.

For example, prolonged exposure to pathogens, which invokes the inflammation response, has been shown to entirely disrupt the metabolic processes. Various studies have shown that Type II diabetics have chronic inflammation which further supports the relationship between the two states. One main cause for such inflammation can be histamine levels. An excess of histamine can produce this prolonged inflammation and disrupt the aforementioned balance between metabolic response and inflammatory response. Amylase, which prevents the release of histamine from Mast cells, can help ameliorate this chronic inflammation by reducing the basal levels of histamine in patients with metabolic disorders. Overall, the role of Amylase in downregulating histamine levels can work on various levels, both direct and indirect, to help patients with metabolic disorders such as Type II diabetes. Histamine elevates the level of key molecules such as cortisol and resistin, both of which lead to insulin resistance and therefore Type II diabetes. A reduction in histamine levels can help reduce the levels of these signaling molecules. Histamine also has a direct influence on Type II diabetics since it produces an over-expressed inflammation response which leads to metabolic disorders. Reducing histamine levels will alleviate such a response.

Mast cell stabilization degranulation, in addition to releasing histamine also causes the release of other inflammatory cytokines such as TNF alpha, IL-Beta which cause inflammation and insulin resistance. Stabilization of Mast cells has been shown to prevent the development of both Type I and Type II diabetes and a reduction in inflammatory cytokines helps preserve beta cell function.

Role of Amylase in Immune Response

Patients with Type II diabetes (and other metabolic disorders) are immuno-compromised. This means that their immune system is not as robust as a healthy individual's and that they are more susceptible to diseases and other disorders. There is evidence that suggests that Type II diabetes can lead to a weakened and/or dysregulated immune system. Additionally, patients with Type II diabetes exhibit high blood sugar, an environment that is highly favorable for bacterial growth which causes additional immune stress.

It has been shown that Mast cell degranulation releases factors that cause T cells to differentiate into the TH1 subtype. Consequently, stabilizing Mast cells will help maintain a healthy ratio of TH1 to TH2 cells. Diabetics and obese individuals typically have a much greater ratio of TH1 to TH2 T cells which makes them more susceptible to viral infections and complications from infections.

A previously unknown interaction between human pancreatic alpha-Amylase and IgE, a peptide implicated in chronic inflammation is disclosed. It is postulated that the low levels of serum human pancreatic alpha-Amylase present in individuals with CF is one of the causes that leads to respiratory failure and pulmonary disease. While the current standard treatment involves supplementation of digestive enzymes the alpha-Amylase in these supplements is usually derived from pigs or bacteria, never human. The divergence of the two Amylase genes occurred after the divergence of pigs and humans. It is postulated that the structure of the alpha-Amylase between species is different enough as to not have the same effect in sequestering IgE. While the starch digesting enzymatic function of Amylase is preserved in all the alpha-Amylases, the primary sequence homology between human pancreatic alpha-Amylase and microbial alpha-Amylase is very low (˜10 to 20%). Between human and porcine alpha-Amylase, the homology is much higher (˜90%). While the topological structure is similar in all cases and the primary enzymatic function (catalysis and digestion of starch and other polysaccharides) is preserved, it is likely that there are slight differences that affect secondary functions of the enzyme. Additionally, computational studies conducted have provided evidence to support this notion. These many differences between porcine, microbial, and human alpha-Amylase lead us to believe that supplementation of pancreatic digestive enzymes in current therapy regimens will not have the same effect as supplementation of human pancreatic alpha-Amylase.

By supplementing human pancreatic alpha-Amylase in individuals with CF, inflammation can be suppressed through the sequestration of IgE, a prominent and well-studied trigger of Mast cells. Given mast cell's role in promoting the inflammatory response, sequestration of IgE seems like an ideal therapeutic target. Human pancreatic alpha-Amylase's ability to sequester IgE through is illustrated herein. The preceding examples can be repeated with similar success by substituting the generically or specifically described reactants and/or operating conditions of this invention for those used in the preceding examples.

Note that in the specification and claims, “about” or “approximately” means within twenty percent (20%) of the numerical amount cited.

The term “a” as used herein means one or more.

Amylase and Amylase mimetic therapies can be formulated in a pharmaceutical composition for administration to a mammalian patient or may be delivered directly.

As used herein, a “pharmaceutical composition” includes an active agent and a pharmaceutically acceptable carrier, excipient or diluent.

The phrase “pharmaceutically acceptable” refers to molecular entities and compositions that are physiologically tolerable and do not typically produce a severe allergic or similar untoward reaction when administered to a mammal. Preferably, as used herein, the term “pharmaceutically acceptable” means approved by a regulatory agency of the Federal or a state government or listed in the U.S. Pharmacopoeia or other generally recognized pharmacopoeia for use in animals, and more particular in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or other aqueous solutions, saline solutions, aqueous dextrose and glycerol solutions are preferably employed as carriers, particularly for injectable solutions. Suitable pharmaceutical carriers are described in “Remington's Pharmaceutical Sciences” by E. W. Martin.

For human therapy, the pharmaceutical compositions, including the active agents, will be prepared in accordance with good manufacturing process (GIMP) standards as set by the Food & Drug Administration (FDA). Quality assurance (QA) and quality control (QC) standards will include testing for purity and function, homogeneity and function, and/or other standard measures.

In order to treat an IgE mediated inflammatory response or other inflammatory response as indicated in FIGS. 4-8 and/or the symptoms arising therefrom, the Amylase, its homologues or analogues or peptide fragments or peptide mimetics or pharmaceutical composition thereof is administered by any route that will permit delivery of the active agent to the affected cells. Since it is believed that Amylase, its homologues or analogues or peptide fragments or peptide mimetics do not harm normal cells, systemic administration of the active agent is acceptable. Preferably, administration is subcutaneous, intramuscular, intraperitoneally and also including, but not limited to, inhalation, intra-arteriole, intravenous, intradermal, topically, orally, perenteral, intraventricular, and intracranial administration. Alternatively, the active agent may be delivered locally to the system or the affected cells by any suitable means.

In therapeutic treatments of the invention, a therapeutically effective amount of the pharmaceutical composition is administered to a mammalian patient. As used herein, the term “therapeutically effective amount” means an amount sufficient to reduce by at least about 15 percent, preferably by at least 50 percent, more preferably by at least 90 percent, and most preferably prevent, a clinically significant metric or deficit in the activity, function and response of the patient. Specifically, a therapeutically effective amount will cause one or more of the following: decreased IgE activity; decreased cortisol levels; stabilized insulin levels; decreased proinflammatory cytokines, decreased proinflammatory interleukins, decreased AGE and or AGE-RAGE complexes, decreased Reactive Oxygen Species, decreased mucous production, or a decrease in any other markers as discussed herein or that would be known to one of ordinary skill in the art as it relates to CF, diabetes, metabolic X syndrome; hyperglycemia, autism, Alzheimer's disease, inflammation or cancer. The frequency and dosage of the therapy can be titrated by the ordinary physician or veterinarian using standard dose-to-response techniques that are well known in the art.

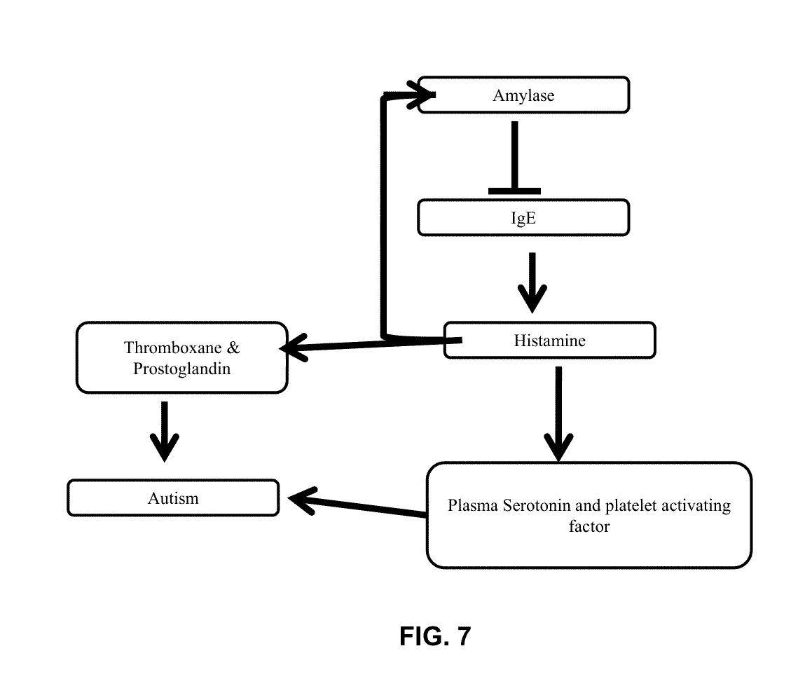

Referring now to FIG. 7, Histamine increases plasma serotonin and platelet activating factor. Histamine activates Thromboxane and prostaglandin. Thromboxane, prostaglandin, plasma serotonin and platelet activating factor are all implicated in the causation of autism.

As noted above, certain embodiments of the present invention involve the use of human alpha-Amylase, homologues thereof, analogues thereof, peptides thereof or peptide mimetics in a therapy as an efficacious treatment of the above mentioned conditions, and/or disorders or diseases or other cells in vitro described herein or as known to one of ordinary skill in the art. In particular, a pharmaceutically effective amount of a compound as disclosed herein or a pharmaceutical composition comprising the compound for treatment or modulation of inflammatory molecules or disorders or disease or symptoms as disclosed or related to symptoms produced thereby is administered to a mammalian patient. Preferably, from about 0.1-10 mg/kg per day, and more preferably from about 1-8 mg/kg per day, and most preferably from about 2-6 mg/kg per day of the pharmaceutical composition is administered to a patient.

A topical application of a composition of the present invention may be administered in a cosmetic amount or in a therapeutically effective dose. The amount of the compound actually administered in therapeutic settings may typically be determined by a physician, in the light of relevant circumstances, including the condition to be treated, the chosen route of administration, the actual compound administered, the age, weight, and response of the individual patient, the severity of the patient's symptoms, and the like. In cosmetic settings, the amount to be applied is selected to achieve a desired cosmetic effect.

The cosmetic compositions of this invention are to be administered topically. The pharmaceutical compositions of this invention are to be administered topically, transdermally or systemically such as orally or by injection.

According to one embodiment of such a cosmetic composition, the amylase or amylase derivative compound is usually a minor component (from about 0.001 to about 20% by weight or preferably from about 0.01 to about 10% by weight) with the remainder being various vehicles or carriers and processing aids helpful for forming the desired dosing form.

Topical cosmetic forms and topical pharmaceutical dosing forms can include lotions, shampoos, soaks, gels, creams, ointments and pastes. Lotions commonly employ a water or alcohol base. Gels are semi-solid emulsions or suspensions. Creams generally contain a significant proportion of water in their base while ointments and creams are commonly more oil-based.

Liquid forms, such as lotions suitable for topical administration or for cosmetic application, may include a suitable aqueous or non-aqueous vehicle with buffers, suspending and dispensing agents, thickeners, penetration enhancers, and the like. Solid forms such as creams or pastes or the like may include, for example, any of the following ingredients, water, oil, alcohol or grease as a substrate with surfactant, polymers such as polyethylene glycol, thickeners, solids and the like. Liquid or solid formulations may include enhanced delivery technologies such as liposomes, microsomes, microsponges and the like.

The above-described components for liquid, semisolid and solid topical compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

When pharmaceutical compositions are to be administered transdermally they typically are employed as liquid solutions or as gels. In these settings the concentration of compounds of the present invention range from about 0.1% to about 20%, and preferably from about 0.1% to about 10%, of the composition with the remainder being aqueous mixed or non-aqueous vehicle, such as alcohols and the like, suspending agents, gelling agents, surfactant, and the like. Examples of suitable such materials are described below.

The peptide-containing compositions of this invention can also be administered in sustained release transdermal forms or from transdermal sustained release drug delivery systems. A description of representative sustained release materials can be found in the incorporated materials in Remington's Pharmaceutical Sciences.

According to one embodiment of the present invention, a topical application such as a spray is useful in the treatment of allergic inflammation, basal cell carcinoma and other inflammations of the skin associated with elevated IgE. The use of the composition on the skin is useful for treating cancers and metastases stemming therefrom.

The compositions for systemic administration include compositions for oral administration, that is liquids and solids, and compositions for injection.

Compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical occupant. Typical unit dosage forms include profiled, premeasured ampules or syringes of the liquid compositions or pills, tablets, capsules or the like in the case of solid compositions. According to one embodiment, a composition of the present invention is usually a minor component (from about 0.01 to about 20% by weight or preferably from about 0.1 to about 15% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an occupant such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

According to another embodiment, injectable compositions are typically based upon injectable sterile saline or phosphate-buffered saline or other injectable carriers known in the art. A compound of the present invention in such compositions is typically a minor component, 0.1-30% by weight with the remainder being the injectable carrier and the like.

The above-described components for orally administrable or injectable compositions are merely representative. Other materials as well as processing techniques and the like are set forth in the part of Remington's Pharmaceutical Sciences noted above. In addition, delivery systems such as disclosed in U.S. Pat. Nos. 8,062,668 and 8,357,400 and artificial pancreases are contemplated as useful delivery mechanism with the compositions as disclosed herein.

The following formulation examples illustrate representative cosmetic and pharmaceutical compositions of this invention. The present invention, however, is not limited to the following pharmaceutical compositions.

Additionally, a composition of the present invention is expected to effectively inhibit the release of cytokines, such as TNF-alpha, IL-6, IL-1 which may be related to the activation of IgE. Such a composition is useful for treating diseases characterized by activation of IgE and production of histamine. Elevated levels of IL-1 and other cytokines are associated with a wide variety of inflammatory conditions, including rheumatoid arthritis, septic shock, erythema nodosum leprosy, septicemia, adult respiratory distress syndrome (ARDS), inflammatory bowel disease (IBD), uveitis, damage from ionizing radiation and the like. Injection dose levels for treating inflammatory conditions range from about 0.1 mg/kg/hour to at least 1.2 mg/kg/hour, all for from about 1 to about 200 hours and especially 15 to 100 hours. A preloading bolus of from about 0.1 mg/kg to about 2 g/kg or more may also be administered to achieve adequate steady state levels.

According to one embodiment of the present invention a new paradigm, system, method, compositions and therapy for modulating one or more proinflammatory microenvironments and/or proinflammatory molecules implicated in the development of the inflammatory chronic diseases is disclosed.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. For example, chronic inflammatory conditions of the skin such as Rosacea or acne, but not limited thereto, may also be treated with a compound as disclosed herein The entire disclosures of all references, applications, patents, and publications cited herein are hereby incorporated by reference.

### REFERENCES

Aughsteen A, Abu-Umair M, Mahmoud S. (2005). “Biochemical a analysis of serum pancreatic Amylase and lipase enzymes in patients with type 1 and type 2 diabetes mellitus” SMJ 26:73-77 Barreto S G, Carati C J, Toouli J, Saccone G T P. (2010) “The islet-acinar axis of the pancreas: more than just insulin” Am J Physiol Gastrointest Liver Physiol 299:G10-G22
Becker K E, Ishizaka T, Metzger H, Ishizaka K, Grimley P (1973). “Surgace IgE on Human Basophils during Histamine Release” JEM 138(2): 394-409
Benkel B F, Hickey D A. (1986) “Glucose Repression of Amylase Gene Expression in Drosophila Melanogaster” Genetics 114: 137-144
Boulet A M, Erwin C R, Rutter W J. (1986) “Cell-specific enhancers in the rat exocrine pancreas” Proc. Natl. Acad. Sci. USA 83:3599-3603
Bratanova-Tochkova T K, Cheng H, Daniel S, Gunawardana, Liu Y J, Mulvaney-M, Schermerhorn R, Straub S, Yajima H, Sharp G (2002) “Triggering and Augmentation Mechanism, Granule Pools, and Biphasic Insulin Secretion” Diabetes 50 supplement S83-90
Cleator J H, Zhu W Q, Vaughan D E, Hamm H E. (2006) “Differential regulation of endothelial exocytosis of P-selectin and von Willebrand factor by protease-activated receptors and camp” Blood 107(7): 2763-2744
Dandona P, Freedman D B, Foo Y, Perkins J, Katrak A, Mikhailidis, Rosalki S B, Beckett A G. (1984) “Exocrine pancreatic function in diabetes mellitus” J Clin Pathol. 37:302-306
Danielsson A. (1973) “Effects of Glucose, Insulin, and Glucagon on Amylase Secretion from incubated mouse pancreas” Pfugers Arch. 348:333-342
Frier B M, Faber O K, Binder C, Elliott H L. (1978) “The Effect of Residual Insulin Secretion on Exocrine pancreatic Function in Juvenile-onset Diabetes Mellitus” Diabetologia 14, 301-304
Ishizaka T, Ishizaka K, Orange R P, Austen K F. (1970) “The Capacity of Human Immunoglobulin E to mediate the Release of Histamine and Slow Reacting Substance of Anaphylaxis (SRS-A) from Monkey Lung” Jour. Immunology 104(2): 335-343
Johnson T M, Rosenberf M P, Meisler M H. (1993) “An insulin-responsive element in the pancreatic enhancer of the Amylase gene” J Biol Chem 268(1):464-8
Kalupahana N S, Moustaid-Moussa N, Claycombe K J. (2012) “Immunity as a link between obesity and insulin resistance” Molecular Aspects of Medicine 33(1):26-34
Morton I, Hall J. (1999) “Concise Dictionary of Pharmacological Agents: Properties and Synonyms” Springer p. 16
Nakajima S, Magee D F. (1970) “Inhibition of exocrine pancreatic secretion by glucagon and D-glucose given intravenously” Can J Physio and Pharm. 48(5): 299-305
Nakajima K, Muneyuki T, Munakata H, Kakei M. (2011a) “Revisiting the cardiometabolic relevance of serum Amylase” BMC Research Notes 4:419
Nakajima K, Nemoto T, Muneyuki T, Kakei M, Fuchigami H, Munakata H. (2011 b) “Low Serum Amylase in association with metabolic syndrome and diabetes: A community-based study” Cardiovascular Diaberology 10:34
Russo H M, Wickenheiser K J, Luo W, Ohman M K, Franchi L, Wright A P, Bodary P F, Nunez G, Eitzman D T. (2010) “P-selectin Glycoprotein Ligand-1 Regulates Adhesive Properties of the Endothelium and Leukocyte Trafficking into Adipose Tissue” JAHA 107:388-397
Santilli F, Vazzana N, Liani R, Guagnano M T, Davi G. (2011) “Platelet Activation in obesity and metabolic syndrome” Obesity Reviews 13(1): 27-42
Sato C, Shikata K, Hirota D, Sasaki M, Nishishita S, Miyamoto S, Kodera R, Ogawa D, Tone A, Katsoka H U, Wada J, Kajitani N, Makino H. (2011) “P-Selectin Glycoprotein Ligand-1 Deficiency is Protective Against Obesity-Related Insulin Resistane” Diabetes 60(1): 189-199
Segal D M, Taurog J D, Metzger H (1977) “Dimeric Immunoglobulin E serves as a unit signal for mast cell degranulation” PNAS 74(7):2993-2997
Snapp, K R, Craig R, Herron M, Nelson R D, Stoolman L M, Kansas G S. (1998) “Dimerization of P-selectin Glycoprotein Ligand-1 (PSGL-1) Required for Optimal Recognition of P-Selectin” Journ of Cell Bio. 142(1): 263-270
Soling H D, Unger K O (1972) “The Role of Insulin in the Regulation of alpha-Amylase Synthesis in the Rat Pancreas” Eur. Jour of Clin Inv 2.4:199-212
Swislocki A, Noth R, Hailstone A, Kyger E, Triadafilopoulos. (2004) “Secretin-stimulated Amylase Release into Blood is Impaired in Type 1 Diabetes Mellitus” Horm Metab Res 37:326-330
Thurmond R L (2010) “Histamine in Inflammation” Adv. in exper. med. and bio. 709: 53
Trimble E R, Bruzzonne R, Belin D (1986) “Insulin resistance is accompanied by impairment of Amylase-gene expression in the exocrine pancreas of the obese Zucker rat” Biochem. J. 237, 807-812
Wang J, Shi G P. Mast cell stabilization: novel medication for obesity and diabetes.
Diabetes Metab Res Rev. 2011 November; 27(8):919-24. doi: 10.1002/dmrr.1272.
Wilcox G. (2005) “Insulin and Insulin Resistance” Clin Biochem Rev Vol 26
Yoo J, Kang Y, Lee K, Kim R (2010) “PP-021-07 KTJ406 inhibits IgE-mediated type 1 hypersensitivity response” Jap. Soc. for Immuno. 22:138-141
Adamis, A., & Berman, A. (2008). Immunological mechanisms in the pathogenesis of diabetic retinopathy. Seminars in Immunopathology, 30(2), 65-84. doi:10.1007/s00281-008-0111-x
Aiello, L. P., Pierce, E. A., Foley, E. D., Takagi, H., Chen, H., Riddle, L., Ferrara, N., et at (1995). Suppression of Retinal Neovascularization in Vivo by Inhibition of Vascular Endothelial Growth Factor (VEGF) Using Soluble VEGF-Receptor Chimeric Proteins. Proceedings of the National Academy of Sciences, 92(23), 10457-10461.
Antonetti, D. A., Barber, A. J., Bronson, S. K., Freeman, W. M., Gardner, T. W., Jefferson, L. S., Kester, M., et al. (2006). Diabetic Retinopathy Seeing Beyond Glucose-Induced Microvascular Disease. Diabetes, 55(9), 2401-2411. doi:10.2337/db05-1635
Basta, G., Schmidt, A. M., & De Caterina, R. (2004). Advanced Glycation End Products and Vascular Inflammation: Implications for Accelerated Atherosclerosis in Diabetes. Cardiovascular Research, 63(4), 582-592. doi:10.1016/j.cardiores.2004.05.001
Benbow, S. J., Wallymahmed, M. E., & MacFarlane, I. A. (1998). Diabetic Peripheral Neuropathy and Quality of Life. QJM, 91(11), 733-737. doi:10.1093/qjmed/91.11.733
Bianchi, R., Buyukakilli, B., Brines, M., Sevino, C., Cavaletti, G., Oggioni, N., Lauria, G., et al. (2004). Erythropoietin Both Protects from and Reverses Experimental Diabetic Neuropathy. Proceedings of the National Academy of Sciences of the United States of America, 101(3), 823-828. doi:10.1073/pnas.0307823100
Boulton, A. J. M., Vinik, A. I., Arezzo, J. C., Bril, V., Feldman, E, L., Freeman, R., Malik, R. A., et al. (2005). Diabetic Neuropathies A Statement by the American Diabetes Association. Diabetes Care, 28(4), 956-962. doi:10.2337/diacare.28.4.956
Conti, G., Stoll, G., Scarpini, E., Baron, P. L., Bianchi, R., Livraghi, S., & Scarlato, G. (1997). p 75 Neurotrophin Receptor Induction and Macrophage Infiltration in Peripheral Nerve during Experimental Diabetic Neuropathy: Possible Relevance on Regeneration. Experimental Neurology, 146(1), 206-211. doi:10.1006/exnr.1997.6521
Conti, G., Scarpini, E., Baron, P., Livraghi, S., Tiriticco, M., Bianchi, R., Vedeler, C., et al. (2002). Macrophage infiltration and death in the nerve during the early phases of experimental diabetic neuropathy: a process concomitant with endoneurial induction of IL-1β and p75NTR. Journal of the Neurological Sciences, 195(1), 35-40. doi:10.1016/S0022-510X(01)00684-0
Economic Costs of Diabetes in the U.S. in 2002. (2003). Diabetes Care, 26(3), 917-932. doi:10.2337/diacare.26.3.917
Engelgau, M. M., Geiss, L. S., Saaddine, J. B., Boyle, J. P., Benjamin, S. M., Gregg, E. W., Tierney, E. F., et al. (2004). The Evolving Diabetes Burden in the United States. Annals of Internal Medicine, 140(11), 945-950.
Finne, P., Reunanen, A., Stenman, S., Groop, P.-H., & Grönhagen-Riska, C. (2005). Incidence of End-Stage Renal Disease in Patients With Type 1 Diabetes. JAMA: The Journal of the American Medical Association, 294(14), 1782-1787. doi:10.1001/jama.294.14.1782
Fong, D. S., Aiello, L., Gardner, T. W., King, G. L., Blankenship, G., Cavallerano, J. D., Ferris, F. L., et al. (2004). Retinopathy in Diabetes. Diabetes Care, 27(90001), 84S-87. doi:10.2337/diacare.27.2007.S84
Fowler, M. J. (2008). Microvascular and Macrovascular Complications of Diabetes. Clinical Diabetes, 26(2), 77-82. doi:10.2337/diaclin.26.2.77
Hansson, G. K., Robertson, A.-K. L., & Söderberg-Nauclér, C. (2006). INFLAMMATION AND ATHEROSCLEROSIS. Annual Review of Pathology: Mechanisms of Disease, 1(1), 297-329. doi:10.1146/annurev.pathol.1.110304.100100
Jones, D. A., Abbassi, O., McIntire, L. V., McEver, R. P., & Smith, C. W. (1993). P-selectin mediates neutrophil rolling on histamine-stimulated endothelial cells. Biophysical Journal, 65(4), 1560-1569. doi:10.1016/50006-3495(93)81195-0
Joussen, A. M., Poulaki, V., Mitsiades, N., Kirchhof, B., Koizumi, K., Dohmen, S., & Adamis, A. P. (2002). Nonsteriodal anti-inflammatory drugs prevent early diabetic retinopathy via TNF-α suppression. The FASEB Journal, 16(3), 438-440. doi:10.1096/fj.01-0707fje
Keenan, H. A., Costacou, T., Sun, J. K., Doria, A., Cavellerano, J., Coney, J., Orchard, T. J., et al. (2007). Clinical Factors Associated With Resistance to Microvascular Complications in Diabetic Patients of Extreme Disease Duration The 50-Year Medalist Study. Diabetes Care, 30(8), 1995-1997. doi:10.2337/dc06-2222
Kilpatrick, E. S., Keevil, B. G., Jagger, C., Spooner, R. J., & Small, M. (2000). Determinants of Raised C-reactive Protein Concentration in Type 1 Diabetes. QJM, 93(4), 231-236. doi:10.1093/qjmed/93.4.231
Kowluru, R. A., & Chan, P.-S. (2007). Oxidative Stress and Diabetic Retinopathy. Experimental Diabetes Research, 2007, 1-12. doi:10.1155/2007/43603
Kunisaki, M., Bursell, S. E., Clermont, A. C., Ishii, H., Ballas, L. M., Jirousek, M. R., Umeda, F., et al. (1995). Vitamin E Prevents Diabetes-Induced Abnormal Retinal Blood Flow Via the Diacylglycerol-Protein Kinase C Pathway. American Journal of Physiology—Endocrinology And Metabolism, 269(2), E239-E246.
Laing, S., Swerdlow, A., Slater, S., Burden, A., Morris, A., Waugh, N., Gatling, W., et al. (2003). Mortality from heart disease in a cohort of 23,000 patients with insulin-treated diabetes. Diabetologia, 46(6), 760-765. doi:10.1007/s00125-003-1116-6
Lechleitner, M., Koch, T., Herold, M., Dzien, A., & Hoppichler, F. (2000). Tumour necrosis factor-alpha plasma level in patients with type 1 diabetes mellitus and its association with glycaemic control and cardiovascular risk factors. Journal of Internal Medicine, 248(1), 67-76.
Lenz, O., Fornoni, A., Ijaz, A., & Tejada, T. (2008). Role of Inflammation in Diabetic Nephropathy. Current Diabetes Reviews, 4(1), 10-17. doi: 10.2174/157339908783502361
Libby, P., Ridker, P. M., & Maseri, A. (2002). Inflammation and Atherosclerosis. Circulation, 105(9), 1135-1143. doi:10.1161/hc0902.104353
McHale, J. F., Harari, O. A., Marshall, D., & Haskard, D. O. (1999). Vascular Endothelial Cell Expression of ICAM-1 and VCAM-1 at the Onset of Eliciting Contact Hypersensitivity in Mice: Evidence for a Dominant Role of TNF-A. The Journal of Immunology, 162(3), 1648-1655.
Meng, H., Tonnesen, M. G., Marchese, M. J., Clark, R. A. F., Bahou, W. F., & Gruber, B. L. (1995). Mast cells are potent regulators of endothelial cell adhesion molecule ICAM-1 and VCAM-1 expression. Journal of Cellular Physiology, 165(1), 40-53. doi:10.1002/jcp.1041650106
Miki, I., Kusano, A., Ohta, S., Hanai, N., Otoshi, M., Masaki, S., Sato, S., et al. (1996). Histamine Enhanced the TNF-α-Induced Expression of E-Selectin and ICAM-1 on Vascular Endothelial Cells. Cellular Immunology, 171(2), 285-288. doi:10.1006/cimm.1996.0205
Mora, C., & Navarro, J. F. (2006). Inflammation and diabetic nephropathy. Current Diabetes Reports, 6(6), 463-468.
Packard, R. R. S., & Libby, P. (2008). Inflammation in Atherosclerosis: From Vascular Biology to Biomarker Discovery and Risk Prediction. Clinical Chemistry, 54(1), 24-38. doi:10.1373/clinchem.2007.097360
Paterson, A. D., Rutledge, B. N., Cleary, P. A., Lachin, J. M., & Crow, R. S. (2007). The Effect of Intensive Diabetes Treatment on Resting Heart Rate in Type 1 Diabetes The Diabetes Control and Complications Trial/Epidemiology of Diabetes Interventions and Complications Study. Diabetes Care, 30(8), 2107-2112. doi:10.2337/dc06-1441
Saraheimo, M., Teppo, A.-M., Forsblom, C., Fagerudd, J., Groop, P.-H., & on behalf of the FinnDiane Study Group. (2003). Diabetic nephropathy is associated with low-grade inflammation in Type 1 diabetic patients. Diabetologia, 46(10), 1402-1407. doi:10.1007/s00125-003-1194-5
Schalkwijk, C. G., Poland, D. C. W., van Dijk, W., Kok, A., Emeis, J. J., Dráger, A. M., Doni, A., et al. (1999). Plasma concentration of C-reactive protein is increased in Type I diabetic patients without clinical macroangiopathy and correlates with markers of endothelial dysfunction: evidence for chronic inflammation. Diabetologia, 42(3), 351-357. doi:10.1007/s001250051162
Schmidt, A. M., Hari, O., Chen, J. X., Li, J. F., Crandall, J., Zhang, J., Cao, R., et al. (1995). Advanced glycation endproducts interacting with their endothelial receptor induce expression of vascular cell adhesion molecule-1 (VCAM-1) in cultured human endothelial cells and in mice. A potential mechanism for the accelerated vasculopathy of diabetes. Journal of Clinical Investigation, 96(3), 1395-1403.
Schram, M. T., Chaturvedi, N., Schalkwijk, C., Giorgino, F., Ebeling, P., Fuller, J. H., & Stehouwer, C. D. (2003). Vascular Risk Factors and Markers of Endothelial Function as Determinants of Inflammatory Markers in Type 1 Diabetes The EURODIAB Prospective Complications Study. Diabetes Care, 26(7), 2165-2173. doi:10.2337/diacare.26.7.2165
Schram, M. T., Chaturvedi, N., Schalkwijk, C. G., Fuller, J. H., Stehouwer, C. D. A., & EURODIAB Prospective Complications Study Group. (2005). Markers of inflammation are cross-sectionally associated with microvascular complications and cardiovascular disease in type 1 diabetes-the EURODIAB Prospective Complications Study. Diabetologia, 48(2), 370-378. doi:10.1007/s00125-004-1628-8
Stitt, A. W. (2003). The role of advanced glycation in the pathogenesis of diabetic retinopathy. Experimental and Molecular Pathology, 75(1), 95-108. doi:10.1016/S0014-4800(03)00035-2
Stoll, G., & Bendszus, M. (2006). Inflammation and Atherosclerosis Novel Insights Into Plaque Formation and Destabilization. Stroke, 37(7), 1923-1932. doi:10.1161/01.STR.0000226901.34927.10
Targher, G., Bertolini, L., Zoppini, G., Zenari, L., & Falezza, G. (2005). Increased plasma markers of inflammation and endothelial dysfunction and their association with microvascular complications in Type 1 diabetic patients without clinically manifest macroangiopathy. Diabetic Medicine, 22(8), 999-1004. doi:10.1111/j.1464-5491.2005.01562.x
Thurmond, R. L. (2010). Histamine in inflammation. Springer.

What is claimed is:

1. A method of modulating IgE mediated histamine release from an IgE receptor-positive cell capable of releasing histamine in-vitro or in-vivo comprising:
providing an effective dose of an Amylase peptide or a derivative thereof to the IgE receptor-positive cell in-vitro or in-vivo under conditions that would permit binding of Amylase to free IgE in solution to form an IgE-Amylase binding pair thereby inhibiting the binding of free IgE to the IgE receptor-positive cell.

2. The method of claim 1 wherein the cell is a mast cell, a basophil or an antigen-presenting dendritic cell.

3. The method of claim 1 wherein the Amylase peptide is pancreatic alpha-Amylase.

4. The method of claim 1wherein the Amylase peptide is selected from SEQ ID NO 1-11 or a derivative thereof.

5. The method of claim 4 wherein the Amylase peptide derivative is a composition having at least 90% sequence homology with amino acids 417-427 of SEQ ID NO. 1 and at least 70% sequence homology with the remaining amino acids of SEQ ID NO 1.

6. A method of treating Type I diabetes, Type II diabetes, Obesity, or Insulin Resistance or secondary complications associated therewith including nephropathy, neuropathy, retinopathy or cardiovascular disease in a mammalian subject comprising:
administering to said subject a therapeutically effective amount of an alpha-Amylase peptide or a derivative thereof.

7. The method of claim 6 wherein the alpha-Amylase is a peptide selected from SEQ ID NO 1-11 or a derivative thereof.

8. The method of claim 6 wherein the Amylase peptide derivative is a composition having at least 90% sequence homology with amino acids 417-427 of SEQ ID NO. 1 and at least 70% sequence homology with the remaining amino acids of SEQ ID NO 1.

9. The method of claim 6 wherein treating Type I diabetes or Type II diabetes includes one or more of modulating serum insulin, preserving beta-cells, and weight loss.

10. The method of claim 9 wherein modulating serum insulin includes decreasing insulin levels in the mammalian subject.

11. The method of claim 6 wherein treating Insulin Resistance includes modulating serum blood Amylase with the therapeutically effective amount of an alpha-Amylase peptide administered.

12. A pharmaceutical composition comprising of a peptide selected from SEQ ID NO 1-11 or a derivative thereof or any combination thereof.

13. A method of treating in a mammalian subject a condition accompanied or caused by IgE mediated histamine release from mast cells comprising:
administering to a host in need of such treatment a therapeutically effective amount of the pharmaceutical composition according to claim 12.

14. The method of claim 13 wherein administering is selected from subcutaneous, intramuscular, intraperitoneally, inhalation, intra-arteriole, intravenous, intradermal, topically, oral, perenteral, intraventricular, and intracranial administration.

15. The method of claim 13 wherein a condition accompanied or caused by IgE mediated histamine release includes allergies and Inflammation, Type I Diabetes, Type II Diabetes, Eczema, Asthma, and Atopic Dermatitis.

16. A skin treatment mixture comprising;
saline and a peptide selected from SEQ ID NO 1-11 or a derivative thereof or any combination thereof.
